(19) United States
(12) Patent Application Publication
(10) Pub. No.: US 2025/0051703 A1
(43) Pub. Date: Feb. 13, 2025

(54) EGG OPENER, EX OVO TUMOR XENOGRAFT METHOD, THERAPEUTIC COMPOUND ASSAY, THERAPEUTIC COMPOUND TESTING METHOD VIA EX OVO QUAIL EGG XENOGRAFT ASSAY

(71) Applicant: CHILDREN'S CANCER THERAPY DEVELOPMENT INSTITUTE, Hillsboro, OR (US)

(72) Inventors: Charles KELLER, III, Beaverton, OR (US); Samuel Val RASMUSSEN, Portland, OR (US)

(21) Appl. No.: 18/697,915

(22) PCT Filed: Oct. 7, 2022
(86) PCT No.: PCT/US2022/046097

§ 371 Date: Apr. 2, 2024

Related U.S. Application Data
(60) Provisional application No. 63/253,261, filed on Oct. 7, 2021.

(51) Int. Cl. C12M 3/10; C12M 1/12; C12M 1/32; C12M 1/33; C12N 5/073; G01N 33/50

(52) U.S. Cl. CPC C12M 21/10; C12M 23/12; C12M 25/14; C12M 45/02; C12N 5/0604; G01N 33/5044; C12N 2503/02

(57) ABSTRACT

An egg opener for a predetermined number of eggs contains a frame and a sliding blade holder which simplifies and speeds up embryo transfer to multi-well plates. A method for preparing an ex ovo tumor xenograft contains the steps of: preparing an ex ovo CAM and adding a cell-infused media to the CAM, wherein the cell-infused media comprises tumor cells to form an ex ovo tumor xenograft. An assay for testing a therapeutic compound contains the steps of providing an ex ovo tumor xenograft as described herein, and adding a therapeutic compound to the CAM, preferably via a therapeutic compound infused in a delivery media such as, for example, an osmotic bead. A method herein can test a therapeutic compound via an ex ovo quail egg xenograft assay, and methods and drugs herein may treat Ewing sarcoma, rhabdomyosarcoma and hepatoblastoma.

### CROSS-REFERENCE TO RELATED APPLICATION

The present application is a national phase entry under 35 U.S.C. § 371 of International Application No. PCT/US2022/046097, filed Oct. 7, 2022, which designates the United States of America, which claims priority to U.S. Provisional Application No. 63/253,261, filed Oct. 7, 2021, the entire disclosures of each of these applications are hereby incorporated by reference in their entireties and for all purposes.

### FIELD OF THE INVENTION

The present invention relates to the field of establishment, preparation and use of shell-free (ex ovo) egg assays, and more particularly chorioallantoic membrane (CAM) assays, methods and their use. The invention further relates to techniques and equipment for egg embryo culturing and engineering and more particularly to avian egg embryo culturing and engineering. The present invention also relates to methods for treating Ewing sarcoma, rhabdomyosarcoma, and hepatoblastoma.

### BACKGROUND OF THE INVENTION

Preclinical cancer research ranges from in vitro studies that are inexpensive and not necessarily reflective of the tumor microenvironment to mouse studies that are better models but prohibitively expensive at scale. Previously, shell-free egg assays have been proposed using chicken eggs (see Fisher, “Chick Embryos in Shell-less Culture”, in Tested studies for laboratory teaching, pp. 105-115, Proceedings of the 5^{th} Workshop/Conference of the Association for Biology Laboratory Education, Goldman, et all, cd., 1993; and PCT Publication WO 2005/033300 A1 to Sierra-Honigmann assigned to Cedars-Sanai Medical Center, published on Apr. 14, 2005). Assays employing shelled eggs, including chicken, duck, turkey, goose, quail, pheasant, grouse, ostrich, cmu, cassowary, and kiwi eggs, are also known (see PCT Publication No. WO 2010/054022 A1 to Chen, published on May 14, 2010, all of which are incorporated herein by reference) and employing various chorioallantoic membrane (CAM) assays.

The CAM is a membrane formed by the fusion of the choiron and allantois membrane on embryonic day 5-6 (see Lokman, et al., “Chick chorioallantoic membrane (CAM) assay as an in vivo model to study the effect of newly identified molecules on ovarian cancer invasion and metastasis”. Int J Mol Sci vol. 13, pp. 9959-70 (2012); Ainsworth, et al., “Developmental stages of the Japanese quail”, J Anat, vol. 216, pp. 3-15 (2010), both incorporated herein by reference). During normal development, the CAM will attach to the inside of the eggshell allowing respiration and calcium extraction for the growing embryo.

The typical methods for culturing avian embryos fall into two general categories: in ovo, whereby a small hole cut into the shell gives access to the embryo (see, for example, Handler, et al., “Studies on mortality in chick embryos resulting from implantation of whole blood and blood fractions from patients and animals with neoplastic disease”, Proc Natl Acad Sci USA, vol. 48, pp. 1549-73 (1962) incorporated herein by reference), and ex ovo with the embryo transferred to a container such as a cell culture plate and grown separate from the shell. The chick methods of in ovo incubation, while effective for chick survival, are time consuming and cannot be scaled because of the need for a skilled operator (see, Deryugina & Quigley, “Chick embryo chorioallantoic membrane model systems to study and visualize human tumor cell metastasis”, Histochem Cell Biol vol. 130, pp. 1119-30 (2008), incorporated herein by reference). For ex ovo approach, another group has reported a culture method for Japanese quail that is novel but also time intensive and unsuitable for automation in multi-well plates (see, Subauste, et al., “Evaluation of metastatic and angiogenic potentials of human colon carcinoma cells in chick embryo model systems”, Clin Exp Metastasis vol. 26, pp. 1033-47 (2009), and Kato, et al., “Culture System for Bobwhite Quail Embryos from the Blastoderm Stage to Hatching”, J Poultry Sci, pp. 155-58 (2013), both incorporated herein by reference).

Approaches to using the avian eggs and/or CAM assays have had preclinical drug administration (dosing) pharmacokinetic challenges, but in recent years the application of drugs into the quail or chick has been approached by topical and intravenous injection (Vargas, et al., “The chick embryo and its chorioallantoic membrane (CAM) for the in vivo evaluation of drug delivery systems”, Adv Drug Deliv. Rev. vol. 59, pp. 1162-76 (2007), incorporated herein by reference), and even intraperitoneally. Such methods again do not lend themselves to automation because of the need of a skilled operator to make the injections.

In addition, it has been found that topical application and even injection may not result in a precise concentration of delivered drug or compound. It has been found that in a topical application the drug may be diluted by an unknown and varying degree and further may spread through the embryo unevenly.

As avian embryotic cultures are not immunocompromised but are instead immune-tolerant, CAM experiments may be conducted for a variety of cancers. However, known CAM methods are very low-throughput as considerable technician expertise is required and there is significant technician-to-technician variability. Thus, the inventors are not aware of any medium-throughput or high-throughput CAM preparation methods, nor any medium-throughput or high-throughput CAM assays reported for analyzing either adult or pediatric cancers. Sec, e.g., CAM via chicken embryos available from INOVATION, La Tronche, France (https://www.inovotion.com/our-technology/a-unique-in-vivo-technology), which employs in ovo assays requiring significant manual manipulation and preparation. This is because for known ex ovo CAM assays, a rate-limiting step is caused by the technical difficulty to transfer of fertilized egg contents with an intact yolk to maintain the embryo viability. To date, such a step requires a skilled artisan to carefully make the transfer, and even with a trained technician this results in significant breakage and variability, all of which reduces throughput, speed and reliability.

CAM assays are also known to grow tumor cells on a 3D scaffold and to detect drug efficacy in, for example, Abraham, et al. “Evasion mechanisms to Igf1r inhibition in rhabdomyosarcoma”, Mol. Cancer There., vol. 10, pp. 697-707 (2011). In this paper, the fertilized quail eggs were washed, dried, sterilized, and incubated at 37.4° C. for 3 days (E3). Forceps were used to remove a small part of the egg shell and the contents of the egg were transferred to a well of a 6-well plate. At E6, 1×10⁶ aleovar rhabdomyosarcoma cells grown on a 3D scaffold (3D Biotek) were added to the chorioallantoic membrane (CAM). Cells of this primary tumor culture also harbor a genetically engineered luciferase gene allowing their detection and quantification. The day following xenotransplantation, 20 μL of complete medium containing 10 μmol/L NVP-AEW541 or 100 μmol/L imatinib was added to the cells. Three days after adding the drug to the cells, 400 μL of 1.5 mg/mL luciferin diluted in PBS was added dropwise to the surface of the CAM. After 30 minutes, the quail embryo was imaged.

Chick embryos are well-established for CAM studies, and chick development is well-documented. Chick embryos typically require 20-21 days for full gestation, and a standardized cell culture plate may only contain 1-2 chicken embryos. Thus, it has now been recognized that the use of chicken embryos results in automation difficulties. However, the developmental stages of other avian species such as Japanese quail are also well-studied from early development to old age. See, e.g., Japanese quail (Coturnix japonica) as a laboratory animal model, Huss, et al., Lab Animal, vol. 37, No. 11, pp. 513-19, 2008, herein incorporated by reference in its entirety.

Thus, it has been found by the inventors that employing quail eggs in a CAM assay possess certain advantages over regular chicken eggs. For example, it has been found that quail eggs may further possess additional advantages such as being cheaper when purchased in bulk, well-sized to fit into a standard six-well dish/plate, and/or that quail eggs lead to easier automation. In a specific example, it has been found that typically only 1-2 chicken embryos would fit on a standard-sized cell culture plate, whereas 6 quail embryos fit on the same-sized plate.

Correlations between, for example quail embryos and chicken embryos are made by comparing literature sources of chicken embryo mass and blood volume with measured quail embryo growth over the same Hamburger and Hamilton stages (see, Mueller, et al., Sturkie's Avian Physiology, (Elsevier, New York, 2015), incorporated herein by reference), where the assumption is that the ratio of blood to body mass is the same during comparable growth stages.

Accordingly, there remains a need for more cost-effective methods and equipment for preparing, performing and using assays, especially CAM assays. There also remains a need for assays for testing drug concentration ranges and assays for rapid testing of ex ovo patient xenografts. There also exists a need for an assay comparable to murine in vivo xenografts, but which is faster and may be performed at a lower cost. There remains a need for an assay for assessing drug toxicity in eggs, and especially quail eggs. There remains a need for an assay employing transgenic eggs, and especially transgenic quail eggs, which allows rapid quantitative and qualitative testing using luminescent markers. There remains a need for an assay which quickly and cost-effectively detects the safety of drugs, and especially the kidney safety of drugs.

There further remains a need for an efficient method and equipment for providing a shell-free CAM assay, especially a shell-free quail egg CAM assay, ready for automated testing. There remains a need for a shell-free CAM assay validated with reporter systems based on imaging analysis, especially luminescent image analysis. There remains a need for an ex ovo assay, especially an ex ovo CAM assay for testing the efficacy and/or toxicity of single drug concentrations.

### SUMMARY OF THE INVENTION

In an embodiment of the invention herein, an egg opener for a predetermined number of eggs contains a frame and a sliding blade holder. The frame contains a predetermined number of egg holders, each egg holder equipped for securing a single egg. The sliding blade holder contains a blade; or a predetermined number of blades; or wherein the predetermined number of blades is equal to the predetermined number of eggs. When the sliding blade holder slides along the frame, the blade cuts each egg within the predetermined number of egg holders.

Without intending to be limited by theory, it is believed that the egg opener herein may result in more efficient embryo transfer into the wells of a multi-well plate, may reduce damage to embryos during transfer, may reduce the time required to transfer embryos into the wells of a multi-well plate, may be automated, and may have other benefits as well.

In an embodiment of the invention herein, a method for preparing an ex ovo tumor xenograft contains the steps of: preparing an ex ovo CAM and adding a cell-infused media to the CAM, wherein the cell-infused media comprises tumor cells to form an ex ovo tumor xenograft.

It is also believed that the method for preparing an ex ovo tumor xenograft herein may be very effective, efficient, and/or may be automated to reduce the time required to prepare such xenografts.

In an embodiment of the invention herein, an assay for testing a therapeutic compound contains the steps of providing an ex ovo tumor xenograft as described herein, and adding a therapeutic compound to the CAM.

Without intending to be limited by theory it is believed that the assay for testing a therapeutic compound herein may lend itself to easy automation, may provide accurate and/or speedy results, and/or may be easy and/or cheap.

In an embodiment of the invention herein, a method for testing a therapeutic compound via an ex ovo quail egg xenograft assay comprising the steps of transferring an embryo to a well in a plate, subjecting the embryo to a CAM formation incubation of from about 60 hours to about 144 hours to develop a CAM, wherein the CAM comprises a CAM surface, culturing a plurality of cells, suspending the plurality of cells in a media to form a cell-infused media, adding the cell-infused media to a scaffold, creating a superficial injury on the CAM surface, placing a ring on the CAM surface, wherein the ring at least partially encloses the superficial injury, and placing the scaffold in the ring and on the CAM surface in contact with the superficial injury, where the scaffold fits within the ring. The method herein further contains the steps of combining a delivery media with a therapeutic compound, placing the delivery media in the ring and on top of the scaffold, subjecting the CAM to an experimental incubation, administering a marker to a marker location selected from the well, the embryo, the CAM and a combination thereof, and detecting the marker.

It is also believed that the method for testing a therapeutic compound herein may be relatively easy, quick, cheap, and/or lend itself to partial or full automation.

In an embodiment herein, a method for treating Ewing sarcoma includes the step of administering to a patient an effective amount of a VEGFR inhibitor, a poly-kinase inhibitor, a EGFR inhibitor, and a combination thereof; or cediranib, erlotinib, and a combination thereof.

Without intending to be limited by theory, it is believed that, based on the data presented herein, an effective amount of these compounds may form the basis for an effective treatment for Ewing sarcoma.

In an embodiment herein, a method for treating rhabdomyosarcoma comprising the step of administering to a patient an effective amount of a PI3K/mTOR inhibitor and a combination thereof; or BEZ235.

Without intending to be limited by theory, it is believed that, based on the data presented herein, an effective amount of these compounds may form the basis for an effective treatment for rhabdomyosarcoma.

In an embodiment herein, a method for treating hepatoblastoma comprising the step of administering to a patient an effective amount of a PLK inhibitor and a combination thereof; or PLK1, volasertib and a combination thereof.

Without intending to be limited by theory, it is believed that, based on the data presented herein, an effective amount of these compounds may form the basis for an effective treatment for hepatoblastoma.

### BRIEF DESCRIPTION OF THE DRAWINGS

FIG. 1 shows an embodiment of a quail egg xenograft tumor method of the present invention;

FIG. 2 shows a detailed schematic drawing of an embodiment of an egg opener of the present invention;

FIG. 3 shows a schematic drawing of an egg opener before use with the handle and sliding blade holder at the beginning position at one end of the rails;

FIG. 4 shows a schematic drawing of the egg opener of FIG. 3 after use, with the handle and sliding blade holder at the opposite end of the rails;

FIG. 5 shows a photograph of an actual egg opener in the before use position;

FIG. 6 shows a graph comparing the percentage of viable quail embryos transferred into a 6-well plate using a manual transfer method and the egg opener device of FIG. 5;

FIG. 7 shows a graph of the percentage of intact embryo transfers over a period of 2.5 month period;

FIG. 8 is a graph showing the number of viable embryos as a function of time of the in ovo incubation;

FIG. 9 shows a photo of a CAM developing in a well of a plate. The quail embryo can be seen as well as the developing network of blood vessels;

FIG. 10 shows a picture of a 3D scaffold as seen in FIG. 1 Step 6;

FIG. 11 and FIG. 12 show cross-sections of a stained CAM;

FIG. 13 shows a representative pseudo-colorized image of viable, luciferase-expressing tumor cells 72 hours after xenografting on the CAM;

FIG. 14 shows the results of the CAM assay employing murine rhabdomyosarcoma cells treated with a therapeutic compound BEZ235;

FIG. 15 shows histology slides stained with H&E of embryonic quail;

FIG. 16A shows a dose-response curve of % viability of HB243 & HB282 in vitro v. volasertib concentration;

FIG. 16B shows a dose-response curve of % viability of HepG2 in vitro v. volasertib concentration;

FIG. 16C shows a dose-response curve of % viability of HB243 & HB282 ex ovo v. volasertib concentration;

FIG. 16D shows a dose-response curve of % viability of HepG2 ex ovo v. volasertib concentration;

FIG. 16E shows % survival of murine xenografts of HB243 v. time with and without drug;

FIG. 16F shows % survival of murine xenografts of HepG2 v. time with and without drug;

FIG. 16G shows the experimental timeline for drug dosing in the murine xenograft studies of FIG. 16E-16F;

FIG. 17A shows a dose-response curve in lumens v. IR820 concentration;

FIG. 17B shows an IR820 diffusion curve in lumens v. time;

FIG. 18A shows a MZ1 dose-response curve tested against a murine melanoma cell line, B16F10 in a CAM assay;

FIG. 18B shows a graph of dabrafenib and trametinib tested alone and in combination against B16F10 in a CAM assay;

FIG. 18C shows a lapatinib dose-response curve as tested against a BT474 xenograft CAM assay;

FIG. 18D shows a trastuzumab dose-response curve as tested against a BT474 xenograft CAM assay;

FIG. 18E shows a MZ1 dose-response curve as tested against a SF8628 xenograft CAM assay;

FIG. 18F shows a panobinostat dose-response curve as tested against a SF8628 xenograft CAM assay;

FIG. 18G shows a IL3Ra2 ADC dose-response curve as tested against a SF8628 xenograft CAM assay;

FIG. 18H shows a MZ1 dose-response curve as tested against a HepG2 xenograft CAM assay;

FIG. 18I shows various levels of Volasertib tested against HepG2;

FIG. 18J shows Bez235 efficacy as tested against a u48484 xenograft CAM assay;

FIG. 19 illustrates cell number vs luminescence for HepG2Glo tumor modules;

FIG. 20 illustrates the diffusion of IR820 small molecule dye from tumor modules;

FIG. 21 illustrates ex ovo patient-derived xenograft; and

FIG. 22 illustrates blood compartments of quail embryos and total blood volume for quail eggs.

The figures herein are for illustrative purposes only and are not necessarily drawn to scale. In the figures, any error bars indicate±the standard error of the mean unless otherwise indicated.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, 60% humidity, sanitary conditions, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

As used herein, the term “plate” indicates any container used to contain the embryo, and is typically a standardized, standard-sized multi-well cell culture plate, such as, for example, a 6-well plate readily-available from multiple vendors around the world. Each well is typically shaped and sized so as to contain a single embryo. These plates are typically optically-transparent plastic or sometimes glass.

Typically most, if not all steps of the procedures and/or steps described herein will take place in a sterile environment unless otherwise specifically stated.

In an embodiment herein, we present an approach to increased CAM assay throughput in the preclinical prioritization of anti-cancer compounds via an automation-ready plate, such as a 6-well plate format.

Without intending to be limited by theory, it is believed that the CAM provides an excellent hypervascularized platform for growing a tumor xenograft. It is further believed that these tumor xenografts may then be used for quantitative and/or qualitative testing of therapeutic compound efficacy against the tumor xenograft.

Without intending to be limited by theory it is also believed that quail embryos provide faster results than, for example, chicken embryos. It is known that chicken embryos require from 20-21 days to gestate, while quail embryos only require from 16-20 days. Thus, as the developmental cycle may be about 25% faster, it has been found that the desired assay may be significantly accelerated when using quail embryos.

In addition, it is believed that the use of quail embryos in a CAM assay is much more space-efficient, as quail embryos are smaller, with 6 embryos fitting onto a standard 6-well cell culture plate, whereas only 1-2 chicken embryos may fit on a comparably-sized cell culture plate. Thus, it is believed that quail embryo CAMs more readily lend themselves to automation and efficient space use as compared to chicken CAMs, or CAMs employing larger eggs.

### Egg Cracking and Viability

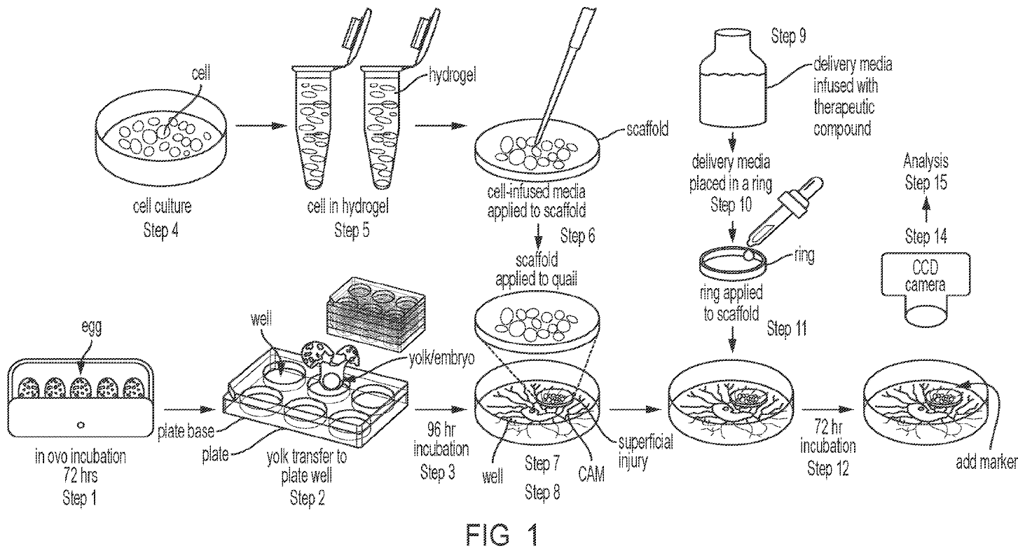

A schematic of an embodiment of a shell-free quail CAM assay herein is presented in FIG. 1. The eggs used herein are typically avian eggs; or chicken eggs, quail eggs, and a combination thereof; or quail eggs; or Japanese quail (Coturnix japonica) eggs. Such eggs are from a variety of vendors worldwide, such as Purely Poultry available (https://www.purelypoultry.com/in Durand, Wisconsin, USA). Without intending to be limited by theory, in an embodiment herein, the eggs used are quail eggs because it is believed that, for example, their size fits easily within standard, commercial 6-well plates, they are readily-available at a relatively low cost, they are sturdy, survive shipping reasonably-well, the quail genome has been sequenced so the quail is a genetically-understood and predictable organism, and/or during development the quail embryo is naturally immune-tolerant.

It is known in the industry that fertilized quail eggs (for example, eggs that have undergone full egg fertilization) may be purchased and stored under standard refrigeration (e.g., from about 1° C. to about 5° C.) for up to about 7 days, or 120 hours, before use. It is also known that these fertilized eggs do not start forming an embryo, growing and gestating until they are subject to an in ovo incubation as described herein.

FIG. 1 shows an embodiment of a quail egg xenograft tumor method of the present invention. In Step 1, fertilized quail eggs are subject to an in ovo (e.g., whole egg) incubation to initiate embryo development. The in ovo incubation may be from about 40 hours to about 100 hours; or from about 45 hours to about 90 hours; or from about 50 hours to about 80 hours; or from about 55 hours to about 72 hours; or from about 58 hours to about 68 hours, at about 37° C. and about 70% relative humidity. More generally, the in ovo incubation is typically at a temperature range of from about 36° C. to about 39° C.; or from about 37° C. to about 38° C.; or from about 37° C. to about 37.7° C. Typically the humidity during the in ovo incubation is from about 45% to about 80%; or from about 50% to about 75%, relative humidity.

While it is possible to include multiple embryos in a single well (see WO 2005/033300 A1 by Sierra-Honigmann, to Cedars-Sainai Medical Center, published on Apr. 14, 2005), in an embodiment herein each well contains a single embryo.

In an embodiment herein, the fertilized quail eggs are periodically rotated during the in ovo fertilization so as to reduce and/or prevent the embryo from attaching to the egg shell.

In Step 2, the fertilized egg yolk (hereinafter referred to as an “embryo”) and at least a portion of the egg white are transferred into a well in a plate; or multi-well plates; or a 6-well plate, to form a filled well in a plate. The plate herein may be a petri dish, or a standardized multi-well plate. Typically each of the wells of the plates will be circular and have a larger diameter than the yolk of the egg to be placed therein. In an embodiment herein, each well has a diameter that is from about 1.1 times to about 5 times; or from about 1.2 times to about 4 times; or from about 1.5 times to about 3 times larger than the diameter of the yolk. in an embodiment herein the wells are optically transparent; or the wells and the plates are optically transparent. In an embodiment herein, each well holds at least about 10 ml; or from about 10 ml to about 100 ml; or from about 12 ml to about 80 ml; or at least 15 ml; or from about 15 ml to about 50 ml.

In a typical ex ovo process known and used prior to the present invention, the transfer of the embryos to the well in the plate is a rate-limiting step requiring considerable expertise. Typically the time to process a single egg using conventional methods involves carefully cutting each egg open with scissors, manually removing the shell pieces with forceps, and carefully pouring the embryo into a dish or a well while avoiding puncture or breakage of the egg yolk, the loss of too much egg white, and/or damage to the embryo. It has been found that the results of this manual process is extremely variable (different amounts of white transferred, different viability ranges, speed variations, etc.), typically requiring from about 5 to about 15 minutes to fill a single 6 well plate, and requires intense focus and precision which wears on and is very stressful for the technician(s) performing the manual procedure. Furthermore, the transfer success rate varies significantly depending on the proficiency of the technician, with observed success rates of from about 30% to about 80%, where success is defined as successfully transferring an embryo without rupturing or otherwise damaging the embryo.

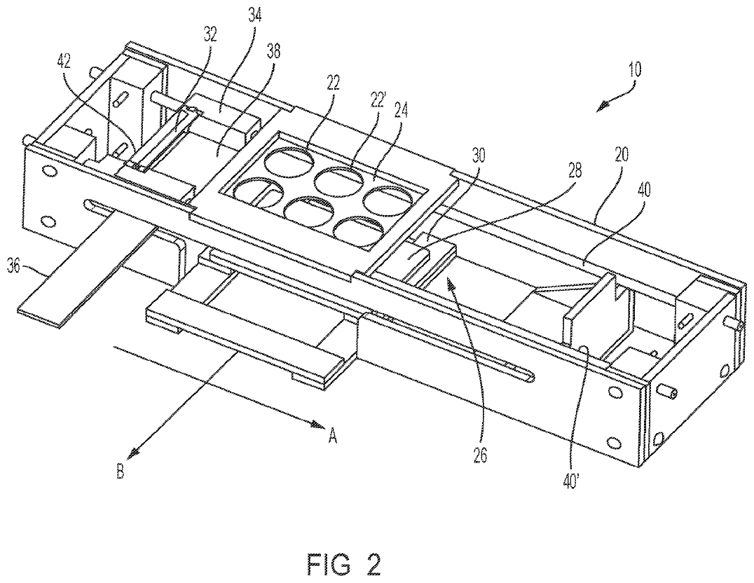

Accordingly, in an embodiment of the invention herein we describe herein a simple egg opener device to process multiple eggs (containing embryos) at a single time to allow the transfer of the embryo contained in the egg into a plate. FIG. 2 shows a detailed schematic drawing of an embodiment of an egg opener, 10, of the present invention, specifically this embodiment is a quail egg opener configured to open 6 quail eggs (see FIG. 1) at a time and deposit the embryos (see FIG. 1) into a corresponding 6 well plate (see. FIG. 1 at Step 2). Without intending to be limited by theory, it is believed that this may be scaled up and/or automated as desired.

In an embodiment herein, the egg opener, 10, holds a predetermined number of eggs; or quail eggs. The egg opener contains a frame, 20, with a predetermined number of egg holders, 22, 22′, etc. each for securing a single egg, which in this embodiment are 6 round holes in an egg holder frame, 24.

In an embodiment herein, the egg holder frame, 22, may be removable, interchangeable, adjustable, etc. such that different-sized eggs and/or different numbers of eggs may be processed with the same egg opener merely by changing or adjusting the egg holder frame, 22. The egg holder frame, 24, is fixed to the frame, 20, during use and does not move relative to the frame, 20 during use. In an embodiment herein, the predetermined number of egg holders is from about 2 egg holders to about 24 egg holders; or from about 4 egg holders to about 12 egg holders; or from about 6 egg holders to about 10 egg holders; or about 6 egg holders. In an embodiment herein the predetermined number of eggs is the same as the predetermined number of egg holders.

Each of the egg holders in the predetermined number of egg holders may be each individually-sized to hold an egg selected from the group consisting of an avian egg; or a chicken egg, a quail egg and a combination thereof; or a quail egg; or a Japanese quail (Coturnix japonica) egg, although typically each of the egg holders in an egg holder frame will be sized to hold the same sized eggs.

In addition to the round holes shown in FIG. 2, it is understood that the egg holders may be, for example, cups which are sized and shaped to allow the eggs sit therein and which may further contain a restraint to secure an egg in each of the egg holders. In an embodiment herein, the restraint secures the eggs in the egg holder even if the frame and/or the egg opener is turned upside down.

The egg opener, 10, in FIG. 2 contains a plate stand, 26, which contains a flat plate, 28, upon which a plate (e.g., a standardized multi-well cell culture plate), typically for quail eggs a 6 well plate (see FIG. 1 at Step 2), securely sits upon during use. The flat plate, 28, is raised up from and attached to a plate stand base, 30. In. FIG. 2, the flat plate, 28, and the plate stand base, 30, are sized and arranged such that a 6 well plate (see FIG. 1 at Step 2) securely fits over the flat piece, 28, and the outside edges of the 6 well plate base (see FIG. 1 at Step 2) sits securely on the plate stand base, 30.

In an embodiment herein multiple egg holder frames and multiple corresponding plates may be present.

A blade, 32, is affixed to a sliding blade holder, 34. In an embodiment herein a plurality of blades are affixed to the sliding blade holder. In an embodiment herein, a single blade is affixed to the blade holder. In an embodiment herein the blade; or plurality of blades, is removable for replacement and/or cleaning.

In FIG. 2, a handle, 36, is attached to the sliding blade holder, 34. The sliding blade holder, 34, further contains an egg fragment catcher, 38, which collects the egg fragments that are cut off by the blade, 32. The sliding blade holder, 34, slides along a pair of rails, 40, 40, which are positioned on the frame, 20, below the egg holder frame, 24.

FIG. 2 shows the position of the handle, 36, and the sliding blade holder, 34, prior to opening the eggs. During use, the eggs are placed in the egg holders, 22, and the egg bottoms protrude from the egg holders, 22. The user then grips the handle, 36, of the sliding blade holder, 34, and moves the handle, 36, in the direction shown by arrow A. This causes the sliding blade holder, 34, to move along the frame, 20, and the rails, 40 and 40′, such that the blade, 32, intersects the eggs and cuts through about the bottom of each egg in the egg holders.

In an embodiment herein the blade, 32, cuts through from about the bottom 10% to about the bottom 30% of the egg; or from about the bottom 15% to about the bottom 25% of the egg; or about the bottom 20% of the egg, as measured according to the height of the egg. Without intending to be limited by theory, it is believed that given the typical shape of a quail egg, this cutting off of the bottom of the egg balances the competing aims of providing a large enough hole in the eggshell (see FIG. 1 at Step 2) through which to drop the embryo and avoiding injury to the yolk. It is also believe that this retains significant amount of egg white (see FIG. 1 at Step 2) to further cushion the dropping of the yolk into the wells of the plate (see FIG. 1 at Step 2) so as to reduce rupture of, or other damage to, the yolk.

In the embodiment of FIG. 2, a wall, 42, at the back of the sliding blade holder, 34, collets the egg shell fragments and therefore prevents the egg shell fragments from escaping from the egg fragment catcher, 38 during use.

The alignment and location of the egg holders, 22, correspond to the alignment and location of the wells of a standardized multi-well cell culture plate; or a 6 well plate, such that when the egg opener, 10, of FIG. 2 is used, the blade, 32, cuts the bottom of the eggs protruding from the egg holders, 22, and the embryos drop into the 6 well plate (see FIG. 1 at Step 2) which is securely resting on the plate stand base, 30. Thus, when a standardized multi-well cell culture plate, such as a 6 well plate, is affixed, typically removably-affixed, to the egg opener, the alignment and location of the predetermined number of egg holders corresponds to the alignment and location of the wells of the standardized multi-well cell culture plate. Therefore, when the blade cuts the (bottoms of the) eggs the egg contents, including the embryo, transfer to the plurality of wells, with typically one embryo per each well.

Once the embryos have dropped into the 6-well plate, then the plate stand, 26, is slid in the direction of arrow B, and the 6-well plate (see FIG. 1 at Step 2) containing the embryos (see FIG. 1 at Step 2); or 6 embryos, is removed from the egg opener, 10.

In order to minimize the potential damage to the embryo and/or rupture of the yolk caused by dropping an embryo into a well of the plate, it is desired to reduce the vertical distance between the blade, 32, and the top of the well (see FIG. 1 at Step 2) and yet provide sufficient clearance to prevent jamming. Thus, in an embodiment herein the distance between the blade and the top of the well is less than about 4 cm; or from about 4 cm to about 0.5 cm; or from about 2 cm to about 0.5 cm.

In a typical embryo transfer process using the egg opener, even an unskilled practitioner may transfer 6 embryos into a 6 well plate in less than 8 minutes; or from about 3 minutes to about 8 minutes, or even faster. Without intending to be limited by theory, it is believed that when employing the egg opener herein, a technician may be able to successfully transfer (e.g., the embryos are viable after transfer) a significantly greater number of embryos into a container or plate at a greater rate. For example, the inventors have found that when employing the device herein even a new technician may process 100 eggs in about 2 hours and 10 minutes, resulting in an average embryo transfer rate of about 1 embryo every 78 seconds, or a 6 well plate in about 7 minutes and 48 seconds. Generally, this is about half the time required using the conventional embryo transfer method.

In addition, it is believed that the transfer success rate may be significantly improved a well. For example, the inventors have found that when employing the device herein a technician may result in a transfer success rate of about 85%, as compared to an average success rate of about 55% when using the conventional embryo transfer method as shown in FIG. 6. Furthermore, it has been found that the variability of this device and process is relatively constant over time, and across various technicians, as shown in FIG. 7 which shows the successful weekly embryo transfer rate of various technicians over a period of 9 weeks. Thus the egg opener and process results in one or more benefits such as significantly higher embryo viability, reduced wastage, lower costs, greater predictability, etc.

In Step 3 (see FIG. 1 at Step 3), the filled well containing the embryo is subject to a CAM formation incubation (see FIG. 1 at Step 3) to grow the CAM within the well. During the CAM formation incubation the CAM develops on the surface of the embryo in the well and the patterning of the embryo occurs. The CAM formation incubation is typically of from about 60 hours to about 144 hours; or from about 72 hours to about 120 hours; or form about 80 hours to about 112 hours; or from about 86 hours to about 100 hours; or about 96 hours then any unfertilized/non-viable egg contents are removed via, for example, by aspiration. The CAM formation incubation is typically at the same conditions as described for the in ovo incubation.

In Step 4 (see FIG. 1 at Step 4), which may occur prior to, and/or concurrently to, the CAM formation incubation in Step 3, at least one cell is cultured (see FIG. 1 at Step 4) to form a plurality of cells. Typically the at least one cell; or plurality of cells is selected from the group of a cell; or a tumor cell; or a liver carcinoma cell; or a hepatoblastoma cell; or a HepG2 human liver hepatoblastoma cancer cell (ATCC HB-8065); or a Ewing sarcoma cell, or a HB234 cell; or a HB282 cell; or a rhabdomyosarcoma cell; or a U48484 (a.k.a., u48484) rhabdomyosarcoma cell; or a SF8628 cell; or a BT474 cell; or a B16F10 cell. In an embodiment herein the cell may be a human cell, an animal cell, and/or a chimeric cell. In an embodiment herein, the cell may be an adult cell (or derived from an adult over 21 years old) or a pediatric cell (or derived from a child—e.g., less than or equal to 21 years old). In an embodiment herein the plurality of cells contains multiple cell types; or multiple tumor cell types. Without intending to be limited by theory, it is believed that since the quail embryo is immunotolerant, virtually any cell line would work in the assay described herein. The cells would typically correspond to the type of cells; or cancer; or condition, which the user intends to study

In Step 5 (see FIG. 1 at Step 5), typically concurrently with the CAM formation incubation in Step 3, the cells are suspended in a media (see FIG. 1 at Step 5); or a gel; or a hydrogel, and a combination thereof to form a cell-infused media. In an embodiment herein, the hydrogel is Hystem-C high collagen hydrogel by Advanced Biomatrix of Carlsbad, California, USA, or Matrigel® (https://www.corning.com/worldwide/en/products/life-sciences/products/surfaces/matrigel-matrix.html) by Corning Life Sciences, Tewksbury, MA, USA

In Step 6 (see FIG. 1 at Step 6), the cell-infused media (see FIG. 1 at Step 6); or gel; or hydrogel, from Step 5 is added to a scaffold (see FIG. 1 at Step 6). In an embodiment herein, the scaffold is an inert glass substrate, such as that available from Lena Biosciences (https://www.lenabio.com/) of Atlanta, GA, USA. The scaffold provides a uniform 3-dimensional space for the cells in the cell-infused media to attach and grow. It is believed that the scaffold significantly helps to decrease assay variability by providing cells with a uniform area to grow and a uniform measurement area, such as for bioluminescent reading of viability. The scaffold (see FIG. 1 at Step 6) may be of a circular shape and sized to fit into the well (see FIG. 1 at Step 2) of the plate (see FIG. 1 at Step 2). In an embodiment herein, when a 6-well plate (see FIG. 1 at Step 2) is being used, the scaffold is a disk of from about 3 mm to about 38 mm in diameter; or from about 4 to about 25 mm in diameter; or from about 4.5 mm to about 20 mm in diameter; or from about 5 to about 10 mm in diameter. Without intending to be limited by theory it is believed that the scaffold surface area should be less than about 35% of the well surface area; or less than about 30% of the well surface area; or less than about 20% of the well surface area; or less than 10% of the well surface area, so as to ensure that the CAM has sufficient available surface area from which to absorb and release gasses for respiration. In an embodiment herein the scaffold is a 6-mm diameter scaffold C-S510-0001 Seedez available from Lena Biosciences, Atlanta GA (https://www.lenabio.com/seedez1.html).

In Step 7 (see FIG. 1 at Step 7), a superficial injury is created on the CAM surface with a sterile instrument, typically a scalpel or other cutting instrument known in the art. In an embodiment herein, the superficial injury is created by placing a dry glass rod against the cam and carefully removing the glass rod.

In Step 8 (see FIG. 1 at Step 8), the scaffold in placed on the CAM; or in contact with the superficial injury; or on the superficial injury; or in the superficial injury, so as to allow the plurality of cells in the scaffold to integrate into the CAM and form an ex ovo xenograft; or, if the cell-infused media contains tumor cells, an ex ovo tumor xenograft.

Traditionally, CAM assays using avian embryos have applied drugs via topical or intravenous injection, which requires a skilled technician to avoid harm to the embryo and/or the CAM. However, in an embodiment of the present invention it has been found that a therapeutic compound may be applied to the CAM via a delivery media which may reduce the chance of embryo damage, provide a more precise dosage, provide a measured/constant diffusion into the scaffold, provide controllable therapeutic compound delivery, etc.

Thus, in Step 9 (see FIG. 1 at Step 9), a delivery media is combined with; or infused with, a therapeutic compound; or a drug; or a water-soluble drug. The delivery media may be a gel, a solid, a liquid and a combination thereof; or a gel, a solid and a combination thereof; or a solid; or a bead; or an osmotic bead, that releasably-delivers the therapeutic compound. In an embodiment herein where the molecular weight of the therapeutic compound is low enough to be absorbed therein, the delivery media is a bead; or an osmotic bead; or a p-10 osmotic bead, such as available from BioRad of Hercules, CA, USA. In an embodiment herein, the delivery media is an osmotic bead infused with PBS+DMSO and the therapeutic compound for about 4 hours. As a control, the delivery media is infused with PBS and DMSO for the same period of time.

The therapeutic compound herein may be any type of drug, small molecule, biological sample (e.g., an antibody, antibody-drug conjugate, a protac (proteolysis-targeting chimera), enzyme, protein, genetic material, vector, plasmid, virus, bacteria, amoeba, etc.), or other compound which the technician or others may wish to test against a particular type of cell or cells (e.g., against the ex ovo xenograft). The biological sample herein may be a wild type or engineered biological sample. It is understood that the therapeutic compound herein may include a single compound or a combination of compounds; may contain a full formulation, or a minimal formulation, may contain a theoretical or suspected therapeutic compound, or a proven therapeutic compound, etc. In an embodiment herein, the therapeutic compound is an anti-cancer therapeutic compound; or a suspected anti-cancer therapeutic compound. In an embodiment herein, the at least one cell is a tumor cell and the therapeutic compound is an anti-cancer therapeutic compound; or a suspected anti-cancer therapeutic compound. In an embodiment herein, the therapeutic compound is selected from the group of a PLK inhibitor, a PI3K/mTOR inhibitor, a VEGFR inhibitor, a poly-kinase inhibitor, an EGFR inhibitor, and a combination thereof; or volasertib, BEZ235, PLK1, cediranib, erlotinib, and a combination thereof.

In Step 10 (see FIG. 1 at Step 10), the delivery media is placed in a ring (see FIG. 1 at Step 10) either before or after the ring is placed on the CAM and around the scaffold—i.e., the order of Step 10 and Step 11 may be interchangeable. In an embodiment herein, the ring is placed on the CAM and around the scaffold and then the delivery media is placed in the ring—i.e., Step 11 (below) is before Step 10.

The ring diameter is typically larger than the scaffold diameter, and the ring height may be greater than the scaffold height, so as to allow the ring to hold a therapeutic compound as a drug depot (see Step 9) as the therapeutic compound diffuses into the scaffold. In an embodiment herein, the ring contains a mesh bottom with holes smaller than the particle size of the delivery media. In another embodiment, the ring lacks a bottom and thus the scaffold sits on the CAM, with the ring surrounding it. In an embodiment herein, the ring provides a barrier around and above the top edge of the scaffold to form the drug depot. In an embodiment herein, the ring is formed of plastic, silicone, metal and a combination thereof; or a plastic, silicone, gold, platinum and a combination thereof; or plastic, silicone, and a combination thereof.

In Step 11 (see FIG. 1 at Step 11), the ring is applied to; or placed on top of, the scaffold. Without intending to be limited by theory, it is believed that the ring holds the beads in fluid contact with; or on, the scaffold so that the drug may gradually diffuse into the scaffold.

In an alternative embodiment herein, the delivery media; or the osmotic beads, (containing either the control or the therapeutic compound) are placed in the ring and on top of the scaffold, which is in turn located on the CAM. Without intending to be limited by theory, it is believed that the therapeutic compound passes from the delivery media to the scaffold and in turn into the CAM where it potentially affects the cells growing on the CAM.

In step 12 (see FIG. 1 at Step 12), the CAM and therapeutic compound; or the well containing the CAM and therapeutic compound, is subjected to an experimental incubation of from about 0 hours to about 144 hours; or from about 24 hours to about 144 hours; or from about 36 hours to about 120 hours; or from about 48 hours to about 96 hours; or from about 56 hours to about 88 hours; or from about 60 hours to about 80 hours; or about 72 hours, to determine how the therapeutic compound affects the development of the cells (or tumor cells; or liver carcinoma cells; or hepatoblastoma cells; or HepG2 human liver hepatoblastoma cancer cells (ATCC HB-8065); or rhabdomyosarcoma cells; or diffuse Intrinsic Pontine Glioma (DIPG) cells; or SF8628 cells; or breast cancer cells; or BT474 cells; or melanoma cells; or B16F10 cells) in the ex ovo xenograft.

In Step 13 (see FIG. 1 at Step 13), a marker, such as, for example, a luminescent marker, a radioactive marker, a fluorescent marker, and a combination thereof; a luminescent marker, a fluorescent marker; and a combination thereof; or a bioluminescent marker; or a fluorescent marker, is administered to a marker location selected from the well, the embryo, the CAM and a combination thereof; or the embryo; or the CAM to help detect the affect (if any) of the therapeutic compound on the cells' (or ex ovo xenograft's) development. Markers ac well-known in the art and available from multiple suppliers worldwide. Those skilled in the art are able to select appropriate markers, depending on various criteria such as the type of cells; or tumor cells, being studied, the type of therapeutic compound being studied, the type of embryo or CAM being used, etc.

In Step 14 (see FIG. 1 at Step 14), the marker is detected; or detected and measured, by the corresponding method such as those known in the art to create experimental data. For example, if the marker is a radioactive marker, then a scintillation counter may be used; for a luminescent and/or fluorescent marker, then a charge-coupled device (CCD) camera may be used (see—see FIG. 1 at Step 14); etc. Typically the marker may provide a result selected from a qualitative result, a quantitative result and a combination thereof; or a quantitative result.

In Step 15 (see FIG. 1 at Step 15), the experimental data is analyzed, typically assisted by a computer and/or an algorithm. For example, the effect of the therapeutic compound may be compared to the control, or the different incubation times are compared, the different incubation conditions are compared, the different dosage levels may be compared, the different therapeutic compounds may be compared, etc. to determine the effect on, for example, the total xenograft cell; or tumor cell; or liver carcinoma cell; or hepatoblastoma cell; or HepG2 human liver hepatoblastoma cancer cell (ATCC HB-8065); or rhabdomyosarcoma cell; or diffuse Intrinsic Pontine Glioma (DIPG) cell; or SF8628 cell; or breast cancer cell; or BT474 cell; or melanoma cells; or B16F10 cell, viability.

Alternatively, to the steps shown in FIG. 1, Steps 6-11, the ring may be placed on the CAM; or on the superficial injury; or in the superficial injury. Typically the ring will at least partially; or completely, enclose the superficial injury. Then the scaffold may be placed within the ring and on the CAM. Typically the scaffold will be in contact with the superficial injury. Typically, the ring will extend around and above the scaffold. The delivery media, infused with the therapeutic compound, may then be placed in the ring on top of the scaffold.

In an embodiment of the method and assay herein, a control is also conducted in parallel to the therapeutic compound. In the control, the therapeutic compound is typically replaced with a placebo and/or simply omitted from the media, CAM, etc. This allows a direct comparison between the activity of the therapeutic compound vs. when no therapeutic compound is present.

In an embodiment herein, no delivery media is used, and the therapeutic compound is delivered directly to the CAM.

In an embodiment herein, the cells; or tumor cells, are not suspended in a media, but are delivered directly to the CAM, for example after forming a plurality of cells.

An embodiment herein includes an assay for testing a therapeutic compound by providing an ex ovo tumor xenograft, especially as described herein, and adding a therapeutic compound to the CAM. It is understood that the steps and embodiments described in the method herein may also be steps or embodiments of the assay herein.

In an embodiment herein, the CAM assay is conducted with quail embryos and the endpoint significance is determined by an unpaired two-tailed t-test with Welch's correction. In such an embodiment, a p-value of less than 0.05 may be considered to be statistically significant.

Without intending to be limited by theory, it is believed that the use of the CAM assay herein may provide a cost-effective and time-efficient screening method to precede and/or minimize the scope and/or need for murine studies, especially for therapeutic compounds such as anti-cancer treatments and/or drug toxicity studies. We believe that the methods and CAM assay described herein may be applicable to a variety of biomedical research endeavors, such as, for example, single-dose therapeutic compound concentrations, therapeutic compound dose-response calculations for multiple concentrations, therapeutic compound dose-response curves, therapeutic compound toxicity tests, validation of marker systems, etc. Furthermore, it is shown that the methods herein may provide quantitative and/or qualitative results.

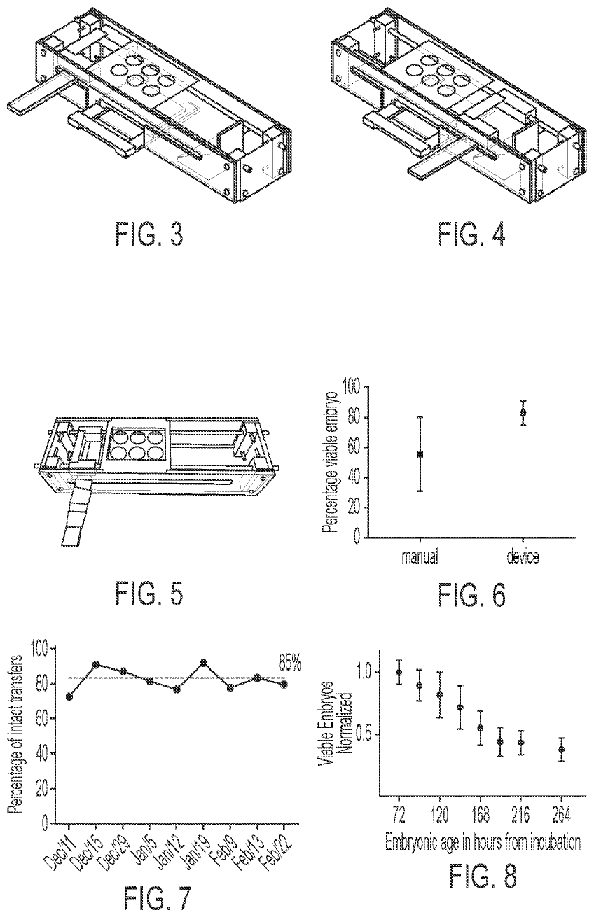

FIG. 3 shows a schematic drawing of an egg opener before use with the handle and sliding blade holder at the beginning position at one end of the rails.

FIG. 4 shows a schematic drawing of the egg opener of FIG. 3 after use, with the handle and sliding blade holder at the opposite end of the rails.

FIG. 5 shows a photograph of an actual egg opener in the before use position.

FIG. 6 shows a graph comparing the percentage of viable quail embryos transferred into a 6-well plate using a manual transfer method and the egg opener device of FIG. 5, where the error bars show the variability of each method. As can be seen, the variability with the manual method is significantly greater than the variability of the method using the egg opener. Also, it is clear that the successful transfer rate is significantly greater with the egg opener method than with the manual method.

FIG. 7 shows a graph of the percentage of intact embryo transfers over a period of 2.5 month period with the same operator. As can be seen, the variability is relatively low, and the percentage of intact transfers is relatively consistent over time.

FIG. 8 is a graph showing the number of viable embryos as a function of time of the in ovo incubation, normalized to 1.0=72 hours in ovo incubation.

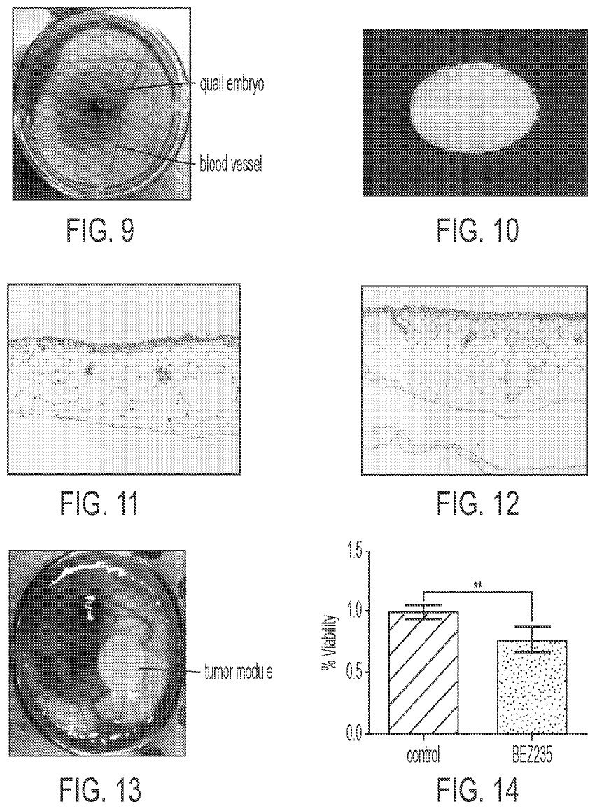

FIG. 9 shows a photo of a CAM developing in a well of a plate. The quail embryo can be seen as well as the developing network of blood vessels.

FIG. 10 shows a picture of a 3D scaffold as seen in FIG. 1 Step 6.

FIG. 11 and FIG. 12 show cross-sections of a stained CAM.

FIG. 13 shows a representative pseudo-colorized image of viable, luciferase-expressing tumor cells 72 hours after xenografting on the CAM.

FIG. 14 shows the results of the CAM assay employing murine rhabdomyosarcoma cells treated with a therapeutic compound BEZ235 (n=6) or vehicle control (n=4). The assay shows a significant difference at p=0.0048.

### Example 1

### Ex Ovo Patient-Derived Xenografts

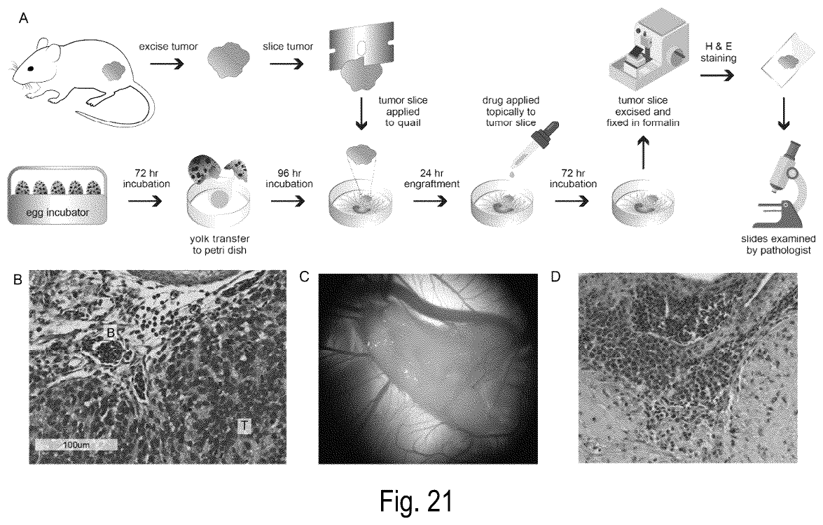

Because generation of patient-derived xenograft using immune-compromised host mice can take 2-7 or more months¹², we tested whether flat sections of patient tumor would engraft on the CAM immune-tolerant platform (FIG. 21). Tumor engraftment (vascularization) from a mouse PDX-derived explant from an Ewing sarcoma occurred within 24 hours and viability was maintained for 96 hours (FIG. 21). Similar results were obtained with an autopsy-derived Ewing sarcoma specimen cryo-preserved, thawed, and engrafted on the CAM (FIG. 21).

### Quail Toxicity Assay

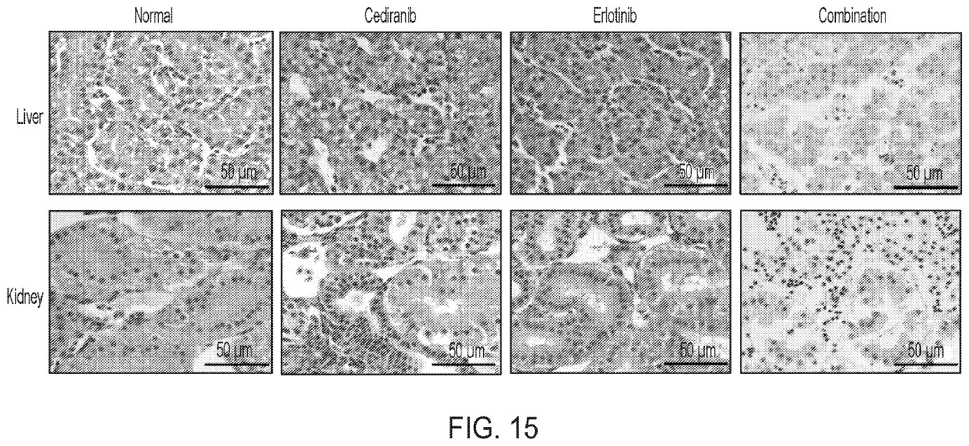

The histology slides were examined by co-pathologist A.M and were found to have significantly more toxicity from the combination of cediranib and erlotinib than either drug alone. Synergistic toxicity was seen in the e10 quail embryo liver and kidney as shown in FIG. 15.

FIG. 15 shows a comparison of liver and kidney for e10 quail dosed with the human clinical Cₘₐₓ of cediranib (VEGFR and poly-kinase inhibitor) and/or erlotinib (EGFR inhibitor) revealing synergistic toxicity in liver and kidney.

Accordingly, it is believed that a VEGFR inhibitor, a poly-kinase inhibitor, a EGFR inhibitor, and a combination thereof; or cediranib, erlotinib, and a combination thereof, may be useful for the treatment of Ewing sarcoma. Thus, an embodiment of the invention herein is a method for treating Ewing sarcoma by administering to a patient an effective amount of a VEGFR inhibitor, a poly-kinase inhibitor, a EGFR inhibitor, and a combination thereof; or cediranib, erlotinib, and a combination thereof. Alternatively, an embodiment of the invention herein includes the use of an effective amount of a VEGFR inhibitor, a poly-kinase inhibitor, a EGFR inhibitor, and a combination thereof; or cediranib, erlotinib, and a combination thereof, in the manufacture of a medicament for the treatment of Ewing sarcoma.

### Example 2

### Quail Tumor Assay

U48484 mouse rhabdomyosarcoma cells were cultured, trypsinized and added to two different vials of hydrogel making a concentration of 10⁶ cells per 50 μl. BEZ235 (cat #S1009, Selleck Chemicals, Houston, TX) in a solution of 0.1% DMF (cat #TS-20673, Thermo Fisher Scientific) in PBS was added to 10⁶ U48484 mouse rhabdomyosarcoma cells mixed with 50 μl hydrogel for a final concentration of 500 nM BEZ235. For untreated eggs, 0.1% DMF in PBS was used as a control. 50 μl of cells/hydrogel/drug mixture were added to each scaffold and incubated for approximately 30-45 minutes at 37° C. and 100% humidity. As detailed above, a superficial injury was created on the chorioallantoic membrane and tumor module containing either drug or control was placed on top. Quail bearing tumor module models were incubated for 72 hours. Add the end of the incubation, 100 μl of PBS containing 1.5 mg of luciferin-d (cat #122799, PerkinElmer) was added to the 3D scaffold, incubated for 10 minutes in the dark, and bioluminescence was measured using a Fluorchem instrument (ProteinSimple, San Jose, CA). The quail were imaged with an 8 minute exposure for total light emission.

Accordingly, it is believed that a PI3K/mTOR inhibitor and a combination thereof; or BEZ235, may be useful for the treatment of rhabdomyosarcoma. Thus, an embodiment of the invention herein is a method for treating rhabdomyosarcoma by administering to a patient an effective amount of a PI3K/mTOR inhibitor and a combination thereof; or BEZ235. Alternatively, an embodiment of the invention herein includes the use of an effective amount of a PI3K/mTOR inhibitor and a combination thereof; or BEZ235, in the manufacture of a medicament for the treatment of rhabdomyosarcoma.

### Example 3

### In Vitro and Ex Ovo Efficacy Testing Across a Drug Concentration Range

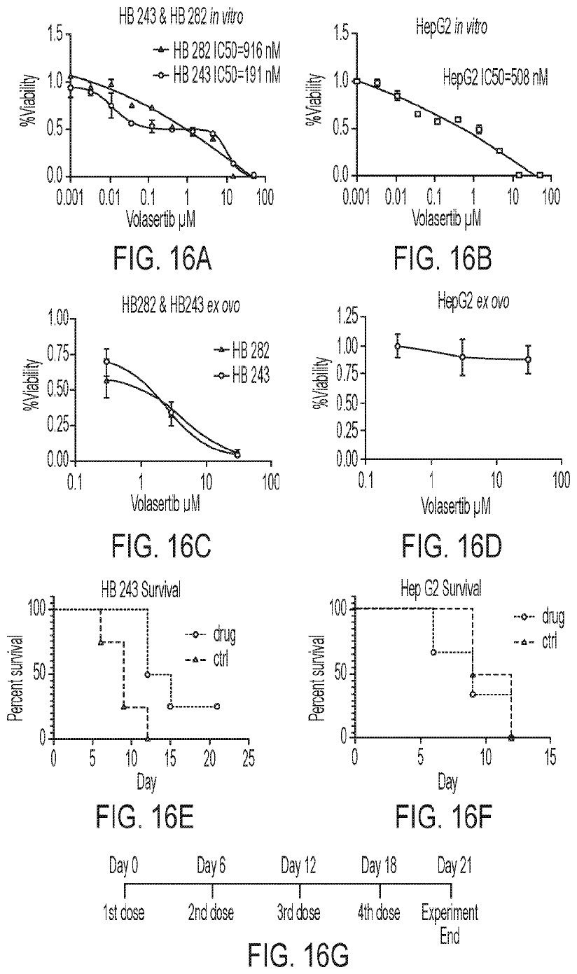

A PLK inhibitor (volasertib) was selected for testing against a range of hepatoblastoma cell lines given that PLK1 is a proposed therapeutic target. The canonical hepatoblastoma cell line HepG2 and patient-derived xenograft (PDX) explanted cell lines HB243 and HB282 were selected as contemporary, robust patient-derived comparators. Volasertib was tested both in vitro and ex ovo against the cell lines across a concentration range. Previous in vitro studies and our own results show that HB282 and HepG2 both were least sensitive to volasertib (IC_50 values 916 nM FIG. 16A and 508 nM in FIG. 16B, respectively), whereas HB243 is considered the most sensitive (IC_50 191 nM in FIG. 16A). For CAM xenografts of these cell cultures, drug delivery is different than in vitro because of CAM vessel washout and embryo drug metabolism; however, in these experiments drug exposure to the engraftment modules was kept constant by osmotic bead drug delivery. Given the small blood volumes of the quail and the desire to automate drug testing, the osmotic bead drug delivery system was chosen and validated for consistent small-molecular research as the most cost-effective method to have a defined drug exposure/drug delivery over time. Ex ovo, HB243 and HB282 were congruent to in vitro studies with a 50% tumor growth inhibition (ex ovo IC_50) of 1455 nM and 1040 nM, respectively (FIG. 16C). On the other hand, HepG2 was insensitive at any concentration as shown in FIG. 16D in stark contrast to the in vitro studies.

FIG. 16E shows the % survival of murine xenografts of HB243 (n=4) v. time with and without volasertib. Survival is defined as tumor volume equal to or greater than 0.75 cc. A significant difference is observed between the treatment and control groups with p=0.031, using the Mantel-Cox test.

FIG. 16F shows the % survival of murine xenografts of HepG2 (n=3) v. time with and without volasertib. Survival is defined as tumor volume equal to or greater than 0.75 cc. Survival of HepG2-bearing murine xenografts did not differ between treatment and control groups with p=0.685 using the Mantel-Cox test.

FIG. 16G shows the experimental timeline for drug dosing in the murine xenograft studies of FIG. 16E-16F.

Accordingly, it is believed that a PLK inhibitor and a combination thereof; or volasertib, may be useful for the treatment of hepatoblastoma. Thus, an embodiment of the invention herein is a method for treating hepatoblastoma by administering to a patient an effective amount of a PKI inhibitor and a combination thereof; or volasertib. Alternatively, an embodiment of the invention herein includes the use of an effective amount of a PKI inhibitor and a combination thereof; or volasertib, in the manufacture of a medicament for the treatment of hepatoblastoma.

### Example 4

### Quail Dose Response Assay

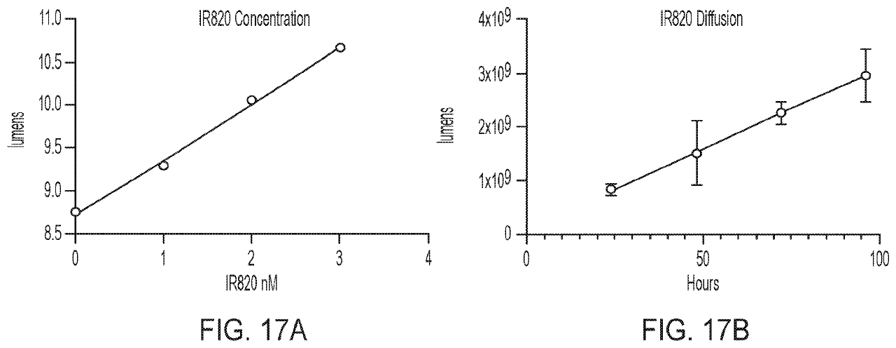

The tumor modules for dose response assay are generated as described above but with 5×10⁵ cells per 50 μl. Drug was dissolved in DMSO for all levels to a final tumor module concentration of vehicle control, 0.3 μM, 3 μM, or 30 μM with n=6. P-10 beads (catalog #1504144, Bio-Rad, Hercules, CA, USA) are soaked in PBS at the concentration of the modules for four hours at room temperature. Approximately 50 μl of bead solution is added to a 9.5×1.5 mm plastic ring placed on top of the tumor module forming a drug depot. The drug depot provided a constant source of drug keeping the tumor module at a constant concentration despite drug leaving the module for the quail, as shown in FIG. 17. The IR820 has an exponential range between 0.001 and 1 μM as shown in the dose-response curve of FIG. 17A in lumens v. IR820 concentration.

FIG. 17B shows an IR820 diffusion curve in lumens v. time indicating that IR820 diffuses through a tumor module at a constant rate and less than 10% of the IR820 diffused through. The quail with tumor module models and drug depots are incubated for 72 hours. Afterwards, 150 μl of 15 mg/ml luciferin (cat #122799, Perkin Elmer) in PBS is added to the modules incubated for 10 minutes and then imaged on an IVIS Lumina (PerkinElmer) for between 15 seconds (for cell lines HepG2) or 1 minute (for cell lines HB243 and HB282).

### Example 5

Validation Assays with Murine and Human Cell Lines

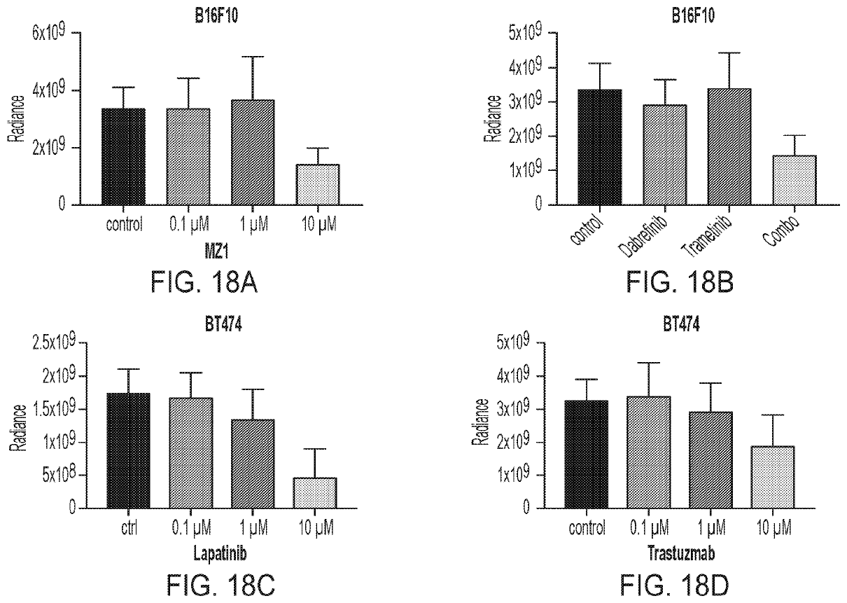

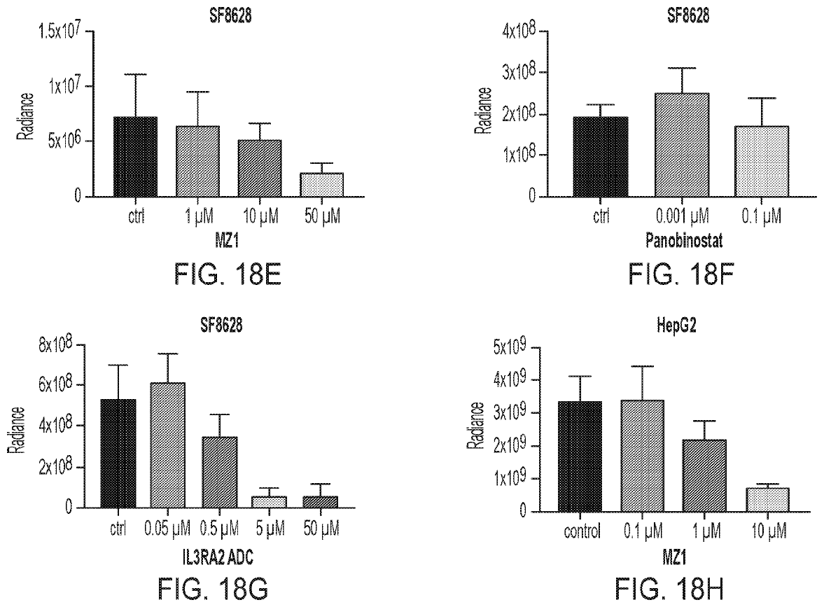

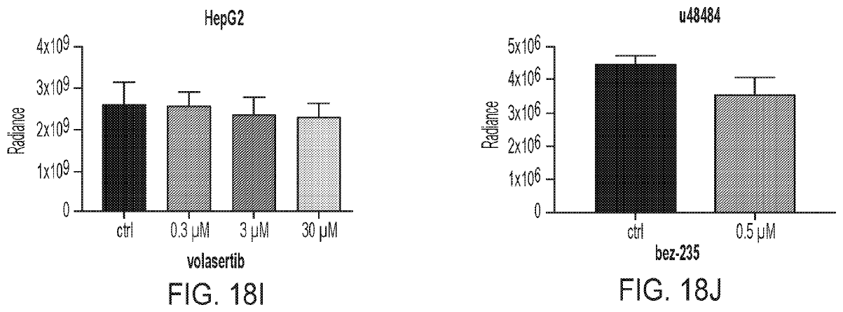

Validation assays are conducted to correlate murine and human cell lines across a range of biologicals and small molecule drugs for pediatric and adult cell lines. B16F10 is a murine melanoma cell line constitutively expressing firefly luciferase (available as cat #BW124734 from Perkin-Elmer, Akron, Ohio USA), which is used to form a CAM xenograft as per the invention herein with the difference of being an adult-type cancer cell line, and radiance reflecting cell viability as impaired by drug. In FIGS. 18A-18J, to analyze drug efficacy on tumor cells, luciferin is added to the scaffold and then imaged by a CCD camera to determine the total xenograft cell viability, with the results indicated as radiance (e.g., steradians/cm²/s) on the Y axis. See also FIG. 1 at Step 14.

FIG. 18A shows a MZ1 (a biological therapeutic that degrades the BRD4 protein available as catalog #36154 from Tocris, Minneapolis, MN, USA), dose-response curve tested against B16F10 in a xenograft CAM assay. The experiment is conducted because of reported disease-specific efficacy correlations shown in human melanoma studies of the exact same drug in vitro. The mouse model corresponds to the human melanoma cell line A375 [See, e.g., Pietrobono, et al., Targeting non-cononical activation of GLII by SOX2-BRD4 transcriptional complex improves the efficacy of HEDGEHOG pathway inhibition in melanoma, Oncogene, 40(22), pp. 3799-814, 2021, doi 10.1038/s41388-021-01783-9; PMID 33958721].

FIG. 18B shows a graph of small molecule inhibitor dabrafenib (available as catalog #S2807 from Selleckchem, Radnor, PA, USA and small molecule inhibitor trametinib (available as catalog #S2673 from Selleckchem, Radnor, PA USA) tested alone and in combination against a B16F10 xenograft in a CAM assay. This experiment is proposed due to efficacy shown in clinical trials [Awada, et al., A phase 2 Clinical Trial of Trametinib and Low-Dose Dabrafenib in Patients with Advanced Pretreated NRAS^{Q6IR/K/L} Mutant Melanoma (TraMel-WT), Cancers, 13 (9), pp. 2010, doi 10.3390/cancers1309210, PMID: 33921947].

As can be seen in FIG. 18A, a dose-response relationship exists for the biological MZ1 when used in the CAM assay herein, whereas in FIG. 18B the effect of the dabrefinib plus trametinib combination in the CAM assay is greater than for either drug individually. Accordingly, it is believed that the present invention may be a useful assay for analyzing drug efficacy for drugs targeting B16F10, A375; murine melanoma, and/or human melanoma.

### Example 6

Validation Assay with Human Breast Cancer Cell Line.

BT474 is a human breast cancer cell line constitutively expressing firefly luciferase (available as catalog #SC-1232 from Cellomics Technology, Halethorpe, MD USA), which is used to form a CAM xenograft as per the invention herein but using an adult-type of cancer cell line instead of a pediatric cell line, most similar to example 5.

FIG. 18C shows a lapatinib (available as catalog #S2111 from Selleckchem, Radnor, PA, USA) dose-response curve as tested against a BT474 xenograft CAM assay. This correlation is based on efficacy shown in BT474 and lapatinib in vitro [see Zhang, et al., Activity of lapatinib is independent of EGFR expression level in HER2-overexpressing breast cancer cells, Mo. Cancer There., 7 (7), pp. 1846-50, 2008, doi 10.1158/1535-7163.MCT-08-0168, PMID: 18644997] and in vivo [Konecny, et al., Activity of the dual kinase inhibitor lapatinib (GW572016) against HER-2-overexpressing and trastuzumab-treated breast cancer cells, Cancer Res., 66 (3), pp. 1630-39, 2006, doi 10.1158/0008-5472.CAN-05-1182, PMID: 16452222].

FIG. 18D shows a trastuzumab (an antibody drug conjugate available as catalog #A2007 from Selleckchem, Radnor, PA, USA) dose-response curve as tested against a BT474 xenograft CAM assay. This experiment is conducted based on the reported efficacy correlation in vitro [Bapat, et al., In Vitro Cytotoxicity of Trastuzumab (Tz) and Se-Trastuzuman (Se-Tz) against Her/2 Breast Cancer Cell Lines JIMT-1 and BT-474, Int. J. Mol. Sci., 22 (9), pp. 4655, doi 10.3390/ijms22094655, PMID: 33925081] and in vivo [PMID: 21876713].

As can be seen in FIGS. 18C and 18D, the CAM assay herein can show a dose-response relationship for the small molecule drug lapatinib and the biological trastuzumab, respectively. Accordingly, it is believed that the CAM assay herein may be useful for analyzing drug efficacy against breast cancer, human breast cancer, and/or adult human breast cancer.

### Example 7

Validation Assay with Human Diffuse Intrinsic Pontine Glioma (DIPG) Cancer Cell Line.

SF8628 is a pediatric human diffuse intrinsic pontine glioma (DIPG) cancer cell line transfected with a non-replicative lentivirus to constitutively expressing firefly luciferase (available as catalog #SCC127 from Sigma-Aldrich, St Louis, MO, USA), which is used to form a CAM xenograft as per the invention herein, which like example 4 shows the use of the assay for a pediatric brain tumor cell line.

FIG. 18E shows a dose-response curve of the biological protein degrader MZ1 (above) as tested against a SF8628 xenograft CAM assay. This experiment is conducted based on efficacy correlations shown using JQ1 (a BRD4 inhibitor that MZ1 is based on) in vitro and in vivo [see Piunti, et al., Therapeutic targeting of polycomb and BET bromodomain proteins in diffuse intrinsic pontine gliomas, Nat. Med., 23 (4), pp. 493-500, 2017, doi 10.1038/nm.4296, PMID: 28263307] as well as BRD4 protac ARV-825 showing efficacy against murine glioma cell line GL261 both in vitro and in vivo [see Yang, et al., A BRD4 PROTAC nanodrug for glioma therapy via the intervention of tumor cells proliferation, apoptosis, and M2 macrophages polarization, Acta. Pharm. Sin. B., 12 (6), pp. 2658-71, 2022, doi 10.1016/j.apsb.2002.02.009, PMID: 35755286].

FIG. 18F shows a panobinostat (available as catalog #S1030 from Selleckchem, Radnor, PA, USA) dose-response curve as tested against a SF8628 xenograft CAM assay. This experiment is based on the efficacy correlation shown against SF8628 in vitro [Cao, Differential kinase activity to ACVRI G328V and R206H mutations with implications to possible TβRI cross-talk in diffuse intrinsic pontine glioma, Sci. Rep., 10 (1), p. 6140, doi 10.1038/s41598-020-63061-0, PMID: 32273545] and in vivo [Tosi, et al., PET, image-guided HDAC inhibition of pediatric diffuse midline glioma improves survival in murine models, Sci. Adv., 6 (30), 2020, doi 10.1126/sciadv.abbv4105, PMID: 32832670] as well as [Grasso et al, Functionally defined therapeutic targets in diffuse intrinsic pontine glioma, Nat Med. 2015 June; 21 (6): 555-9. doi: 10.1038/nm.3855; PMID 25939062].

FIG. 18G shows a IL3Ra2 ADC [available by synthesis from the anti-IL13Ra2 2E10 primary antibody, Cat #WH0003598M1; Sigma-Aldrich, St. Louis, MO, USA, conjugated to duo-carmycin DM via non-cleavable linker (Fab-aMFc-CL-DMD), Cat #AM-202-DD; Moradec LLC., San Diego, CA, USA] dose-response curve as tested against a SF8628 xenograft in a CAM assay. This experiment is conducted because of the efficacy correlation shown in vitro [Lian, et al., Design considerations of an IL13Ra2 antibody-drug conjugate for diffuse intrinsic pontine glioma, Acta. Neuropatol. Commun., 9 (1), pp. 88, 2021, doi 10/1186/s40478-021-01184-9, PMID: 34001278].

As can be seen in FIGS. 18C and 18D, a dose-response relationship can be observed for the small molecule drug panobinostat and the biological IL13Ra2 ADC, respectively. It is believed that the CAM assay herein may therefore be useful for analyzing drug efficacy against pediatric cancer, human pediatric cancer, DIPG cancer, and/or human pediatric DIPG cancer.

### Example 8

Validation Assay with Human Hepatoblastoma Cell Line.

HepG2 is a human pediatric hepatoblastoma cell line transfected with a non-replicative lentivirus to constitutively express firefly luciferase (available as catalog #8065 from ATCC, Manassas, VA, USA), which is used to form a CAM xenograft as per the invention herein most similar to example 4.

FIG. 18H shows a MZ1 (above) dose-response curve as tested against a HepG2 xenograft CAM assay. This experiment is conducted due to the reported efficacy correlation shown against HepG2 in vitro [see, Luo, et al., Profiling of diverse tumor types establishes the broad utility of VHL-based ProTaCs and triages candidate ubiquitin ligases, iScience, 25 (3), 103985, 2022, doi 10.1016/j.sci.2022.103985, PMID: 35295813]. It is noted that JQ1, a Brd4 inhibitor, showed efficacy against Hep3B, a distantly related human hepatocellular carcinoma, in vivo [see, Li, et al., Suppression of BRD4 inhibits human hepatocellular carcinoma by repressing MYC and enhancing BIM expression, Oncotarget, 7 (3), pp. 2462-74, 2016, doi 10.18632/oncotarget.6275, PMID: 26575167].

FIG. 18I shows a volasertib (available as catalog #S2235 from Selleckchem, Radnor, PA, USA) dose-response curve as tested against a HepG2 xenograft CAM assay. This experiment is conducted due to the reported in vitro efficacy indicated in Rasmussen, et al., Preclinical therapeutics ex ovo quail eggs as a biomimetic automation-ready xenograft platform, Sci. Rep., 11 (1), pp. 23302, 2021, doi 10.1038/s41498-021-02509-3, PMID 34857796].

As can be seen in FIGS. 18H and 18I, a dose-response relationship can be observed for the biological MZ1 and the small molecule volasertib, respectively. Accordingly, it is believed that the CAM assay herein may therefore be useful for analyzing drug efficacy against hepatoblastoma, pediatric hepatoblastoma, human hepatoblastoma, and/or human pediatric hepatoblastoma.

### Example 9

Validation Assay with Murine Rhabdomyosarcoma Cell Line.

u48484 is a murine rhabdomyosarcoma cell line constitutively expressing firefly luciferase (available from the originating laboratory as described in Aslam et al, PDGFRβ reverses EphB4 signaling in alveolar rhabdomyosarcoma, Proc Natl Acad Sci USA. 2014 Apr. 29; 111 (17): 6383-8. doi: 10.1073/pnas. 1403608111; PMID 24733895), which is used to form a CAM xenograft as per the invention herein except being a non-human tumor cell line.

FIG. 18J shows small molecule inhibitor Bez235 (available as catalog #S1009 from Selleckchem, Radnor, PA USA) efficacy as tested against a u48484 xenograft CAM assay. This experiment shows the efficacy correlation as reported in Rasmussen et al, Preclinical therapeutics ex ovo quail eggs as a biomimetic automation-ready xenograft platform, Sci Rep. 2021 Dec. 2; 11 (1): 23302. doi: 10.1038/s41598-021-02509-3; PMID 34857796. See also the efficacy correlation shown against rhabdomyosarcoma both in vitro and against u48484 [Ricker, et al., Defining an embryonal rhabdomyosarcoma endotype, Case Reports, Cold Spring Harb. Mol. Case Stud., 6 (2), p. a005066, 2020, doi: 10.1101/mcs.a005066, PMID: 32238403] and in vivo against RD/18, a clone of human rhabdomyosarcoma cell line RD [Manara, et al., NVP-BEZ235 as a new therapeutic option for sarcomas, Clin. Cancer Res., 16 (2), pp. 530-40, 2010, doi 10.1158/1078-0432.CCR-09-0816, PMID: 20068094].

As can be seen in FIG. 18J, a drug-related inhibition of tumor growth can be observed for the small molecule Bez-235. Accordingly, it is believed that the CAM assay herein may therefore be useful for analyzing drug efficacy against rhabdomyosarcoma, murine rhabdomyosarcoma, and/or human rhabdomyosarcoma.

Non-limiting embodiments of the invention herein include:

Embodiment 1) An egg opener for a predetermined number of eggs comprising:
A. a frame, the frame comprising a predetermined number of egg holders, each egg holder equipped for securing a single egg; and
B. a sliding blade holder slidable along the frame, the sliding blade holder comprising a blade; or a predetermined number of blades,
wherein when the sliding blade holder slides along the frame, the blade cuts each egg in the egg holders.

Embodiment 2) The egg opener according to embodiment 1, wherein the predetermined number of eggs is from about 2 eggs to about 24 eggs; or from about 4 eggs to about 12 eggs; or from about 6 eggs to about 10 eggs; or about 6 eggs.

Embodiment 3) The egg opener according to any one of the previous embodiments, wherein the predetermined number of egg holders are each sized to hold an egg selected from the group consisting of an avian egg; or a chicken egg, a quail egg and a combination thereof; or a quail egg; or a Japanese quail (Coturnix japonica) egg.

Embodiment 4) The egg opener according to any one of the previous embodiments, further comprising a standardized multi-well cell culture plate affixed to the egg opener, wherein the alignment and location of the predetermined number of egg holders corresponds to the alignment and location of the wells of the standardized multi-well cell culture plate, and wherein when the blade cuts the eggs the egg contents transfer to the plurality of wells.

Embodiment 5) The egg opener according to any of the previous embodiments, wherein when the blade cuts each egg, the sliding blade holder collects the egg shell fragments.

Embodiment 6) The egg opener according to embodiments 4, wherein the standardized multi-well plate is removably-affixed to the egg opener.

Embodiment 7) A method for preparing an ex ovo tumor xenograft comprising the steps of:
A. preparing an ex ovo CAM; and
B. adding a cell-infused media to the CAM, to form an ex ovo tumor xenograft,
wherein the cell-infused media comprises tumor cells.

Embodiment 8) The method for preparing an ex ovo tumor xenograft according to embodiments 7, further comprising the step of adding the cell-infused media to a scaffold prior to adding the cell-infused media to the CAM.

Embodiment 9) The method for preparing an ex ovo tumor xenograft according to any one of embodiments 7 to 8, further comprising the step of creating a superficial injury to the CAM and placing the cell-infused media in contact with the superficial injury.

Embodiment 10) The method for preparing an ex ovo tumor xenograft according to any one of embodiments 7 to 9, further comprising an experimental incubation wherein the CAM is incubated for from 0 hours to about 144 hours after adding the cell-infused media to the CAM.

Embodiment 11) The method for preparing an ex ovo tumor xenograft according to any one of embodiments 7 to 10, wherein the tumor cells are suspended in a media; or a gel; or a hydrogel and a combination thereof, to form the cell-infused media.

Embodiment 12) An assay for testing a therapeutic compound comprising the steps of:
A. providing an ex ovo tumor xenograft according to the method according to any one of embodiments 7 to 11; and
B. adding a therapeutic compound to the CAM.

Embodiment 13) The assay for testing a therapeutic compound according to embodiments 12, further comprising the steps of:
placing a ring around the scaffold; and
adding the therapeutic compound into the ring.

Embodiment 14) The assay for testing a therapeutic compound according to any one of embodiments 12 or 13, wherein the therapeutic compound is provided in a delivery media; or wherein the delivery media is selected form the group consisting of a gel, a solid, a liquid and a combination thereof; or a gel, a solid and a combination thereof; or a solid; or a bead; or an osmotic bead.

Embodiment 15) The assay for testing a therapeutic compound according to any one of embodiments 12 to 14, comprising an experimental incubation step wherein the CAM is incubated for from 0 hours to about 144 hours after adding the cell-infused media to the CAM.

Embodiment 16) The assay for testing a therapeutic compound according to any one of embodiments 12 to 15, wherein a plurality of different therapeutic compound concentrations are tested on a plurality of CAMs.

Embodiment 17) The assay for testing a therapeutic compound according to any one of embodiments 12 to 16 further comprising the steps of:
adding a marker to the CAM; and
detecting the marker.

Embodiment 18) A method for testing a therapeutic compound via an ex ovo quail egg xenograft assay comprising the steps of:
A. transferring a quail embryo to a well in a standardized multi-well cell culture plate;
B. subjecting the embryo to a CAM formation incubation of from about 60 hours to about 144 hours to develop a CAM, wherein the CAM comprises a CAM surface;
C. culturing at least one cell to form a plurality of cells;
D. suspending the plurality of cells in a media to form a cell-infused media;
E. adding the cell-infused media to a scaffold;
F. creating a superficial injury on the CAM surface;
G. placing a ring on the CAM surface, wherein the ring at least partially encloses the superficial injury;
H. placing the scaffold on the CAM surface in contact with the superficial injury, wherein the scaffold is placed within the ring;
I. combining a delivery media with a therapeutic compound;
J. placing the delivery media in the ring and on top of the scaffold;
K. subjecting the CAM to an experimental incubation;
L. administering a marker to a marker location selected from the well, the embryo, the CAM and a combination thereof; and
M. detecting the marker to generate experimental data.

Embodiment 19) The method for testing a therapeutic compound via an ex ovo quail egg xenograft assay according to embodiment 18, wherein the plurality of cells are a tumor cell; or a liver carcinoma cell; or a hepatoblastoma cell; or a HepG2 human liver hepatoblastoma cancer cell, (ATCC HB-8065); or a Ewing sarcoma cell, or a HB234 cell; or a HB282 cell; or a rhabdomyosarcoma cell; or a U48484 (u48484) rhabdomyosarcoma cell; or a SF8628 cell; or a BT474 cell; or a B16F10 cell.

Embodiment 20) The method for testing a therapeutic compound via an ex ovo quail egg xenograft assay according to any one of embodiments 18 to 19, wherein the media is a gel; or a hydrogel.

Embodiment 21) The method for testing a therapeutic compound via an ex ovo quail egg xenograft assay according to any one of embodiments 18 to 20, wherein the experimental incubation is from about 0 hours to about 144 hours.

Embodiment 22) The method for testing a therapeutic compound via an ex ovo quail egg xenograft assay according to any one of embodiments 18 to 21, wherein the marker is a luminescent marker, a radioactive marker, a fluorescent marker, and a combination thereof; a luminescent marker, a fluorescent marker; and a combination thereof; or a bioluminescent marker; or a fluorescent marker.

Embodiment 23) The method for testing a therapeutic compound via an ex ovo quail egg xenograft assay according to any one of embodiments 18 to 22, further comprising the step of analyzing the experimental data.

Embodiment 24) A method for treating Ewing sarcoma comprising the step of administering to a patient an effective amount of a VEGFR inhibitor, a poly-kinase inhibitor, a EGFR inhibitor, and a combination thereof; or cediranib, erlotinib, and a combination thereof.

Embodiment 25) A method for treating rhabdomyosarcoma comprising the step of administering to a patient an effective amount of a PI3K/mTOR inhibitor and a combination thereof; or BEZ235.

Embodiment 26) A method for treating hepatoblastoma comprising the step of administering to a patient an effective amount of a PLK inhibitor and a combination thereof; or PLK1, volasertib and a combination thereof.

It should be understood that the above only illustrates and describes representative examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

The below is a reproduction of a research paper submitted for publication by the inventors. This research paper was published on Dec. 2, 2021, and may be cited as Rasmussen et al, Preclinical therapeutics ex ovo quail eggs as a biomimetic automation-ready xenograft platform, Sci Rep. 2021 Dec. 2; 11 (1): 23302. doi: 10.1038/s41598-021-02509-3; PMID 34857796

### Preclinical Therapeutics Ex Ovo

### Quail Eggs as a Biomimetic Automation-Ready Xenograft Platform

Samuel V. Rasmussen¹, Noah E. Berlow¹, Lisa Hudson Price¹, Atiya Mansoor¹, Stefano Cairo², Sandra Rugonyi³, Charles Keller^{1#}¹ Children's Cancer Therapy Development Institute, Beaverton, OR 97005 USA² Xentech, Evry FRANCE³ Department of Biomedical Engineering, Oregon Health & Science University, Portland OR 97239 USA^{#}correspondence: Charles Keller M D, 12655 Sw Beaverdam Rd West, Beaverton OR 97005 USA, Tel: 801-232-8038, Fax: 270-675-3313, email: charles@cc-tdi.org

### Acknowledgements

Funding for this work was supported by the Macy Easom Cancer Research Foundation, as well as The Foundation for Addie's Research, Owls for Avery Foundation, Rutledge Foundation, the Super Sam Foundation, the Go4TheGoal Foundation and the Sam Day Foundation.

Histology for this work was done by the OHSU Histopathology Shared Resource.

### Abstract

Preclinical cancer research ranges from in vitro studies that are inexpensive and not necessarily reflective of the tumor microenvironment to mouse studies that are better models but prohibitively expensive at scale. Chorioallantoic membrane (CAM) assays utilizing Japanese quail (Coturnix japonica) are a cost-effective screening method to precede and minimize the scope of murine studies for anti-cancer efficacy and drug toxicity. To increase the throughput of CAM assays we have built and optimized an 11-day platform for processing up to 200 quail eggs per screening to evaluate drug efficacy and drug toxicity caused by a therapeutic. We demonstrate ex ovo concordance with murine in vivo studies, even when the in vitro and in vivo studies diverge, suggesting a role for this quail shell-free CAM xenograft assay in the validation of new anti-cancer agents.

### Introduction

Pediatric cancer has historically limited new drug development as demonstrated by only 10 new agents earning primary childhood cancer FDA approval since 1978. To develop new therapies for rare disease, a cost-effective workflow from basic science target identification to preclinical research and then to clinical investigation is needed. Current preclinical research approaches move from in vitro studies to in vivo murine models; however, most of the time drug response data obtained from in vitro assays fail to be confirmed in vivo. As mouse studies are sometimes prohibitively expensive and time-consuming, taking tens of thousands of dollars and 10 or more weeks to complete, it is crucial to develop innovative cost and time-effective processes to improve the selection of anti-cancer agents to be prioritized for preclinical mouse studies Here we propose a re-examined and optimized the shell-free quail chorioallantoic membrane assay (CAM) as a precursor to mouse preclinical studies^{1,2}.

CAM assays have traditionally utilized chicken (chick) or quail eggs, with chick being used more commonly. Chick CAM models have been employed for the study of angiogenesis, tumor growth and metastasis³⁻⁵. The CAM is a membrane formed by the fusion of the chorion and allantois membrane on embryonic day 5-6 (e5-6)⁶. The membrane will attach to the inside of the eggshell allowing respiration and calcium extraction for the growing embryo. The methods for culturing embryos fall into two categories: in ovo, whereby a small hole cut into the shell gives access to the embryo (for example, the studies performed by Sidney Farber in 1962⁵), or ex ovo with the embryo transferred to a cell culture plate and grown separate from the shell. The chick methods of in ovo incubation, while effective for chick survival, are time consuming and cannot be scaled because of the need for a skilled operator⁷. For ex ovo approach, another group has reported a culture method for Japanese quail that is novel but also time intensive and unsuitable for automation in multi-well plates^{4,8}. Parenthetically, too, the Japanese quail (Coturnix japonica) genome was sequenced in 2016 and is publicly available.

Approaches to using the avian eggs and/or CAM assays have had preclinical drug administration (dosing) pharmacokinetic challenges, but in recent years the application of drugs into the quail or chick has been approached by topical and intravenous injection⁹. To circumvent systemic drug administration, drug can be admixed with tumor cells in an extracellular matrix that is applied to the quail CAM^{1,3}.

CAM experiments have been conducted on a variety of cancers, but to our knowledge no medium- or high-throughput methods have been reported for either adult or pediatric cancers. Herein, we present an approach to increased throughput of CAM assays in the preclinical prioritization of anti-cancer compounds via an automation-ready 6-well plate format.

### Results

### Egg Cracking and Viability

A schematic of the shell-free quail CAM assay is presented in FIG. 1. A rate-limiting step is to transfer egg contents with the yolk intact to a multi-well plate. A simple apparatus to process 6 eggs at a time is shown in FIGS. 2A-C. The total time required to conduct the transfer from in ovo to ex ovo for 100 eggs is approximately 2 hours 10 minutes. This approach has intact-yolk transfer rate of 85% on average as shown in FIG. 2D, with egg content transfer to multi-well plates is consistently achieved as shown in FIG. 2E. A second challenge was to determine the stage of egg development with the least spontaneous embryo death. From our studies, embryo survival decreases to 40-50% survival by embryonic day 10 with most of the losses between embryonic day 4 and day 7 (FIG. 2F); therefore, day 7 (e7) was identified as the best time point for tumor xenografting.

FIG. 1. Schematic for the quail egg drug screening assay. The quail eggs are incubated for 72 hours and then the embryos are transferred to six-well plates. The six-well plates are incubated for 96 hours then any non-viable egg contents are removed. During the quail egg incubation, tumor cells are cultured. After the 96 hour incubation the tumor cells are suspended in hydrogel, added to a 3D scaffold, and applied to the chorioallantoic membrane. A 9 mm ring is placed onto the 3D scaffold and then filled with P-10 osmotic beads soaked and equilibrated in the therapeutic compound to be tested. The quail is then incubated for 72 hours. To analyze drug efficacy on tumor cells, luciferin is added to the scaffold and then imaged by a CCD camera to determine the total xenograft cell viability. Copyright holder Children's Cancer Therapy Development Institute.

FIGS. 2-8. Embryo Transfer to multi-well plates. FIGS. 2-4) drawings of the quail egg opener in the before and after positions for opening quail eggs. FIG. 5) The blade collision device to open the quail eggs and transfer them to the six-well plate. FIG. 6) The quail batches used for the device method varied between 40 and 75 quail eggs per batch with 8 batches done. Three batches were used for the manual forceps method had between 32 and 75 quail eggs each with 3 batches done, error bars are standard deviation. FIG. 7) Number of viable egg yolks transferred to six-well plates weekly over a period of nine weeks. FIG. 8) The age vs survival of the quail embryos used to choose the experiment start date error bars are standard deviation.

### Validation of the Reporter Systems Used

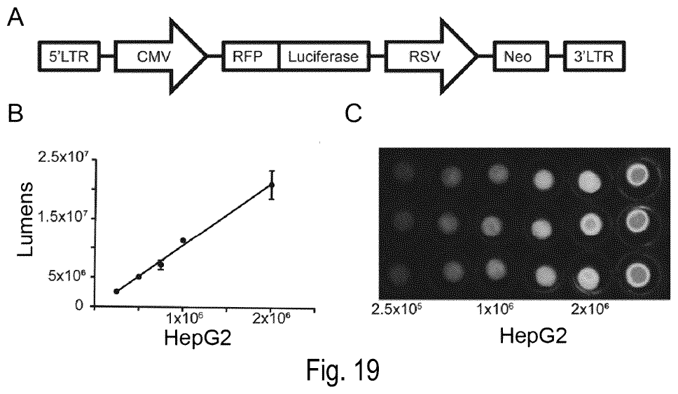

We next confirmed bioluminescence of tumor cells using a commercially-available luciferase-red fluorescent protein (RFP) lentivirus reporter system (FIG. 19). The luminescence was linear with the tumor cell counts of xenograft-ready “modules” consisting of tumor cells embedded in hydrogel on a fiberglass scaffold (FIG. 19). As a cautionary note, for the HepG2 cell line the luminescence and cell number were linear in our range employed but became non-linear at 4×10⁶ cells per tumor module (FIG. 19). A transgenic rhabdomyosarcoma cell culture (U48484²) demonstrated a linear relationship between luminescence and tumor cell number for the range of 1×10⁵ and 1.25×10⁶ cells per module (data not shown). These ranges can be considered narrow however they provide an accurate linear range to compare and are bright enough to ensure visualization by bioluminescence.

### Ex Ovo Efficacy Testing at Single Drug Concentrations

We initially validated our system using the dual PI3K/mTOR inhibitor BEZ235 was tested on the rhabdomyosarcoma cell culture U48484². A 3D scaffold (FIG. 10) was used for the tumor module which was supported by the capillary-rich CAM (FIGS. 11-12). We tested BEZ235 at 500 nM in the hydrogel volume (FIG. 13) and had an observable 20% reduction in U48484 tumor module bioluminescence (tumor cell viability) as shown in FIG. 14. We are cautious to note, however, that the efficacy of BEZ235 could have been decreased by diffusion into the CAM, which led us to pursue a drug depot/osmotic drug release approach described below.

FIGS. 9-14 illustrate the quail xenograft assay. FIG. 9) a quail embryo at e7. FIG. 10) An unused 3D scaffold 9.5 mm in diameter for use as a tumor module. FIGS. 11-12) Cross sections of the CAM. FIG. 13) representative pseudo-colorized image of viable, luciferase-expressing tumor cells 72 hours after xenografting on the CAM. FIG. 14) The assay used murine rhabdomyosarcoma cells treated with BEZ235 (n=6) or vehicle control (n=4). **, significance p=0.0048.

### In Vitro and Ex Ovo Efficacy Testing Across a Drug Concentration Range

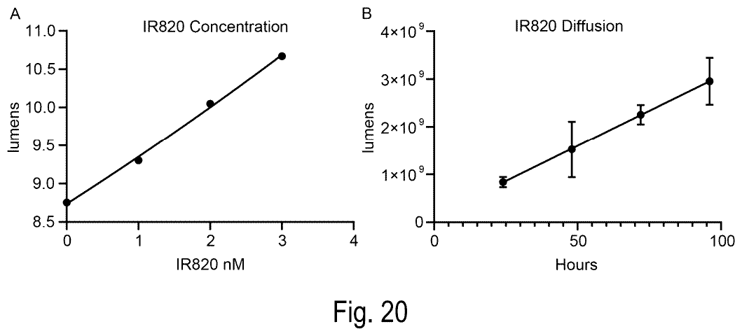

A PLK inhibitor (volasertib) was selected for testing against a range of hepatoblastoma cell lines given that PLK1 is a proposed therapeutic target¹⁰. The canonical hepatoblastoma cell line HepG2 and patient-derived xenograft (PDX) explanted cell lines HB243 and HB282 were selected as contemporary, robust patient-derived comparators¹¹. Volasertib was tested both in vitro and ex ovo against the cell lines across a concentration range. Previous in vitro studies¹⁰ and our own results show that HB282 and HepG2 both were least sensitive to volasertib (IC50 values 916 nM FIG. 4A and 508 nM in FIG. 4B, respectively), whereas HB243 is considered the most sensitive (IC50 282 nM in FIG. 4A). For CAM xenografts of these cell cultures, drug delivery is different than in vitro because of CAM vessel washout and embryo drug metabolism; however, in these experiments drug exposure to the engraftment modules was kept constant by osmotic bead drug delivery. Given the small blood volumes of the quail (FIG. 20) and the desire to automate drug testing, the osmotic bead drug delivery system was chosen and validated for consistent small-molecular research (FIG. 21) as the most cost-effective method to have a defined drug exposure/drug delivery over time. Ex ovo, HB243 and HB282 were congruent to in vitro studies with a 50% tumor growth inhibition (ex ovo IC50) of 1455 nM and 1040 nM, respectively (FIG. 16C). On the other hand, HepG2 was insensitive at any concentration as shown in FIG. 16D in stark contrast to the in vitro studies.

### Murine In Vivo Xenograft Study Comparisons

In cell line-based mouse xenograft studies, HB243 and HepG2 had different responses to volasertib in vivo. HB243 caused a statistically significant reduction in growth relative to the control group as shown in FIG. 16E with the treatment mice surviving to the endpoint an average of 4.5 days longer than control (vehicle). HepG2 had no increase in survival in FIG. 16F with no significant difference between the volasertib-treated cohort and the control cohort. Volasertib resistance in HepG2 could not be attributed to PLK expression or ABC transporter (drug efflux) expression due to no consistent overexpression (Table 2) as we did not find a clear pattern with ABC genes in HepG2 having a consistently higher expression than HB243 and HB282 however some such as ANCG2 were expressed more in HepG2.

FIGS. 16A-16G illustrate comparison of in vitro, quail egg ex ovo xenograft and mouse in vivo xenograft results. FIGS. 16A-16B) In vitro drug curves, each point has n=4 replicates. FIGS. 16C-16D) For the ex ovo drug curves, each concentration has n=4-6 replicates. FIG. 16 E) The murine xenograft survival analyses for HB243 (n=4) defined an event occurrence as tumor volume equal or greater than 0.75 cc, and a significant difference observed between treatment and control groups with a p=0.031 using the Mantel-Cox test. FIG. 16F) The survival of HepG2 bearing mice (n=3) using the same criteria did not have a difference between treatment and control groups with p=. 685. G) Shown is the experiment timeline for drug dosing in mouse xenograft studies.

### Ex Ovo Patient-Derived Xenografts

Because generation of patient-derived xenograft using immune-compromised host mice can take 2-7 or more months¹², we tested whether flat sections of patient tumor would engraft on the CAM immune-tolerant platform (FIG. 21). Tumor engraftment (vascularization) from a mouse PDX-derived explant from an Ewing sarcoma occurred within 24 hours and viability was maintained for 96 hours (FIG. 21). Similar results were obtained with an autopsy-derived Ewing sarcoma specimen cryo-preserved, thawed, and engrafted on the CAM (FIG. 21).

### Quail Toxicity Assay

The histology slides were examined by co-pathologist A.M and were found to have significantly more toxicity from the combination of cediranib and erlotinib than either drug alone. Synergistic toxicity was seen in the e10 quail embryo liver and kidney as shown in FIG. 15.

FIG. 15 illustrates histology slides stained with H&E of embryonic quail. Comparison of liver and kidney for e10 quail dosed with the human clinical Cₘₐₓ of cediranib (VEGFR and poly-kinase inhibitor) and/or erlotinib (EGFR inhibitor) revealing synergistic toxicity in liver and kidney.

### Discussion

Herein we report methods for reliably and reproducibly performing ex ovo drug testing in shell-free quail CAM assay xenografts. We believe this platform brings us close to automation through mechanical systems for transferring egg contents to multi-well plates and we demonstrate the feasibility of single concentration xenograft testing, or xenograft testing across a concentration range (e.g., the ex ovo IC_50). When culturing the quail ex ovo we noticed a majority of the die off between e4 and e7 most likely due to the shock of transferring the embryo to the six well plate. At e7 the chorioallantoic membrane had fully formed and providing a capillary rich surface to support the tumor module. We tested volasertib in mouse xenografts and compared the in vivo results to the ex ovo quail xenograft results and observed that HepG2 continued to be resistant in concordance with the ex ovo experiments and in contrast to the in vitro experiments. HB243 had a statistically significant reduction in growth in concordance to both the sensitivity of HB243 to volasertib in the ex ovo and in vitro experiments. The resistance of HepG2 to volasertib in vivo and ex ovo but not in vitro deserves further analysis. We conducted RNA sequencing that could not discern ABC transporters overexpression as the cause of resistance. Our validation studies were conducted solely for hepatoblastoma, but parallel remain to be done for other pediatric and adult cancers. The readiness in which patient xenografts engraft to the CAM is an exciting opportunity for further research.

Future directions will include addressing the pharmacokinetic considerations for efficacy and toxicity models in lieu of our described approach of hydrogel-based tumor modules as a single compartment model. Given the small blood volumes of the quail CAM, and the changing blood volumes from e7 to e11, serial micro-sampling approaches will require careful but worthwhile optimizations.

### Methods

### Quail Preparation

All experiments were conducted in accordance with Children's Cancer Therapy Development Institute policies and all relevant guidelines. Coturnix japonica eggs were purchased from Boyd's Bird Company (Eagle Creek, OR) and PurelyPoultry (Fremont, WI), stored at 4° C. for 120 hours and then incubated at 37° C. and 70% humidity for approximately 72 hours. Quail eggs were opened by our mechanical device (FIG. 2) and the embryos and white transferred to a six-well plate. Six-well plates were incubated for 96 hours (embryonic day 7, e7) at which point unfertilized/non-viable egg contents were removed and viable quail used for assays.

### Cell Lines

All cell lines were obtained as de-identified samples. HepG2 human liver carcinoma cells (ATCC, HB-8065) were transfected with lentiviral particles containing RFP, luciferase, and neomycin resistance following manufacturer's instructions (cat #LVP677, Gentarget, San Diego, CA). Reporter-transfected cells were purified using 800 nM G418 antibiotic selection 24 hours, flow sorted, then antibiotic selected again with the resulting cell line stably expressing RFP and luciferase. Cells were maintained in DMEM (cat #11990573, Thermo Fisher Scientific, Waltham, MA) with 10% FBS (Thermo Fisher Scientific, cat #10437036) and 1% penicillin-streptomycin (cat #15140122, Thermo Fisher Scientific). HB282 was received from co-author Stefano Cairo [Xentech] and transfected with a lentiviral particle containing RFP, luciferase, and puromycin resistance following manufacturer's instructions (cat #LVP674, Gentarget). HB282 followed the previously listed selection process but with a puromycin (cat #73342, Stemcell Technologies, Cambridge, MA) concentration of 2 μg/ml. HB282 was cultured in ADMEM, 10% FBS, 1% penicillin-streptomycin, 1% L-glutamine. We received HB243 transfected with GFP and luciferase from co-author Stefano Cairo [Xentech]. Previously characterized U48484 murine alveolar rhabdomyosarcoma (aRMS) cells which stably express a luciferase reporter transgene were maintained in DMEM, 10% FBS, and 1% penicillin-streptomycin 2.

### Luminescence Calibration

All experiments were conducted in accordance with Children's Cancer Therapy Development Institute policies and all relevant guidelines. To generate a standard curve for luminescence, 7 different cell densities ranging from 0 to 4×10⁶ of HepG2Glo were suspended in Hydrogel-c (cat #GS313, ESI-BIO, Alameda, CA) and 50 μl were added to 9.5 mm diameter sterilized fiberglass 3D mesh in FIG. 19 (cat #SC-S510-0001, Lena Bioscience, Atlanta, GA) according to manufacturer's protocol in a 24 well plate. Two hundred μl of luciferin-d (cat #122799, Perkin Elmer, Waltham, MA) at 15 mg/ml diluted in PBS was added to each well, incubated for 10 minutes, and then imaged using a UVP Biospectrum 600 (Analytik Jena US LLC, Upland, CA). Luminescence readings were processed using Prism 8.0 (Graphpad Software, San Diego, CA, https://www.graphpad.com/scientific-software/prism/). Luminescence calibration was performed in the same manner for U48484 mouse aRMS cells with the range of cells from 0 to 1.5×10⁶ cells per module.

Patient-Derived Xenograft onto the CAM

A diagram of the procedure is presented in FIG. 21 with all experiments carried out performed after receiving approval from the institutional animal care committee (IACUC) at Children's Cancer Therapy Development Institute. Samples were collected from patients who had given informed consent and enrolled in the CuReFAST tumor banking study approved of by the Children's Cancer Therapy Development Institute's Institutional Review Board (Advarra, protocol #cc-TDI-IRB-1). An Ewing sarcoma patient derived xenograft from a 23 year-old male surgically implanted to a NSG mouse at Jax laboratory (model ID TM01617) was removed from the mouse, encased in 2% agarose at 37° C. and sliced to 1 mm thick. The tumor slices were removed from the agarose and applied to an injury site on e7 quail embryos. Injury was created by placing a dry glass rod against the cam and carefully removing the glass rod. Twenty μl of Matrigel was added on top of the tumor slice to covering the slice to avoid any drying. The tumor engrafted for 96 hours and was then removed and fixed in formalin. Samples were then sectioned and H & E stained at Oregon Health & Science University (OHSU) Histopathology Shared Resource.

### Chick to Quail Blood Volume Comparison

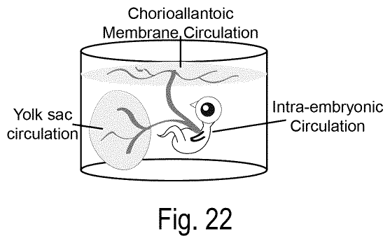

All experiments were conducted in accordance with Children's Cancer Therapy Development Institute policies and all relevant guidelines. In order to develop pharmacokinetic approximations for the quail we compared literature sources for chicken embryo mass and blood volume growth to our measured quail embryo growth over the same Hamburger and Hamilton stages FIG. 22. We assumed that the ratio of blood volume to body mass would be the same during the same growth stages.

### Quail Tumor Assay

All experiments were conducted in accordance with Children's Cancer Therapy Development Institute policies and all relevant guidelines. U48484 mouse rhabdomyosarcoma cells were cultured, trypsinized and added to two different vials of hydrogel making a concentration of 10⁶ cells per 50 μl. BEZ235 (cat #S1009, Selleck Chemicals, Houston, TX) in a solution of 0.1% DMF (cat #TS-20673, Thermo Fisher Scientific) in PBS was added to 10⁶ U48484 mouse rhabdomyosarcoma cells mixed with 50 μl hydrogel for a final concentration of 500 nM BEZ235. For untreated eggs, 0.1% DMF in PBS was used as a control. 50 μl of cells/hydrogel/drug mixture were added to each scaffold and incubated for approximately 30-45 minutes at 37° C. and 100% humidity. As detailed above, a superficial injury was created on the chorioallantoic membrane and tumor module containing either drug or control was placed on top. Quail bearing tumor module models were incubated for 72 hours. Add the end of the incubation, 100 μl of PBS containing 1.5 mg of luciferin-d (cat #122799, Perkin Elmer) was added to the 3D scaffold, incubated for 10 minutes in the dark, and bioluminescence was measured using a Fluorchem instrument (Protein Simple, San Jose, CA). The quail were imaged with an 8 minute exposure for total light emission.

### Quail Dose Response Assay

All experiments were conducted in accordance with Children's Cancer Therapy Development Institute policies and all relevant guidelines. The tumor modules for dose response assay were generated as described above but with 5×10⁵ cells per 50 μl. Drug was dissolved in DMSO for all levels to a final tumor module concentration of vehicle control, 0.3 μM, 3 μM, or 30 μM with n=6. P-10 beads (cat #1504144, Bio-Rad, Hercules, CA) were soaked in PBS at the concentration of the modules for four hours at room temperature. Approximately 50 μl of bead solution was added to a 9.5×1.5 mm plastic ring placed on top of the tumor module forming a drug depot. The drug depot provided a constant source of drug keeping the tumor module at a constant concentration despite drug leaving the module for the quail, as shown in FIG. 20. The IR820 had an exponential range between 0.001 and 1 μM as shown in FIG. 19 (section A). The IR820 diffused through a tumor module at a constant rate and less than 10% of the IR820 diffused through as shown in FIG. 19. The quail with tumor module models and drug depots were incubated for 72 hours. Afterwards, 150 μl of 15 mg/ml luciferin (cat #122799, Perkin Elmer) in PBS was added to the modules incubated for 10 minutes and then imaged on an IVIS Lumina (Perkin Elmer) for between 15 seconds (for cell lines HepG2) or 1 minute (for cell lines HB243 and HB282).

### In Vivo Mice Experiment

All studies in mice were performed after receiving approval from the institutional animal care and use committee (IACUC) at Children's Cancer Therapy Development Institute and in accordance with ARRIVE Guidelines. HepG2 and HB243 were suspended in Matrigel and injected into n=10 eight week-old female nod scid gamma mice per cell line xenograft (Charles River, Hollister, CA, NOD.CB17-Prkde^{scid}/NCrCrl) with 2×10⁶ cells per 100 μl injection. The dosing schedule shown in FIG. 16G began after reaching 0.25 cubic centimeters in volume with mice that did not develop tumors excluded. Volasertib (cat #S2235, Selleck Chem) was suspended in 3.75% DMSO and corn oil (cat #C8267, Sigma-Aldrich, St Louis, MO) at a concentration of 1.5 mg/ml with 100 μl injected intraperitoneally with mice chosen for the drug and control group randomly selected to minimize selection bias. The mice were imaged by intraperitoneal injection of luciferin-d according to manufacturer's instructions. The tumors were measured using calipers every 3 days and the equation for volume was V=L×W×H×0.5(π/6). At the study end or if the tumors reached 1.5 cc the tumors were removed and measured.

### Quail Toxicity Assay

All experiments were conducted in accordance with Children's Cancer Therapy Development Institute policies and all relevant guidelines. Cediranib (Cat #S1017, Selleck Chemicals LLC, Houston, TX) and erlotinib (Cat #S7786) were purchased from Selleck Chemicals and reconstituted in dimethyl sulfoxide (DMSO) following the manufacturers recommendations and diluted to 10 mM stock concentration.

Fertilized Japanese quail eggs were incubated and plated as described previously. Quail were allowed to grow ex ovo in six-well plates until the quail had passed the patterning phase (e8 based on plating date). Each experimental arm was assigned n=4 viable quail and treated with one of four experimental conditions: vehicle, cediranib, ertoltinib, and cediranib+erlotinib. Dosages provided to quail were based on maximum clinically-achievable serum concentrations (Cₘₐₓ) in human patients, specifically 42 ng/ml for cediranib¹³ and 1.3 μg/mL for erlotinib¹⁴. Stock concentrations were diluted in phosphate-buffered saline (PBS) to the respective target concentrations and to a final volume of 25 μL per agent. DMSO for vehicle was set at the DMSO volume used in the cediranib+erlotinib combination (8.4 μL DMSO).

Vehicle and diluted agents were subsequently applied dropwise to the quail chorioallantoic membrane. Quail were photographed at 0 hours, 24 hours, and 48 hours. Remaining viable quail (n=4 vehicle, n=3 cediranib, n=3 erlotinib, n=4 combination) were sacrificed 48 hours after dosing and fixed in 10% formalin for 24 hours. Fixed quail were transported to the OHSU Histology core, paraffin embedded, sectioned in coronal orientation, and stained with hematoxylin and eosin. Stained images were analyzed by pathologist co-author A.M. for kidney and liver histopathology looking for signs of normal versus abnormal development (FIG. 15).

### Sequencing of Samples

Each cell line was grown to 80% confluency, trypsinized, and snap frozen. RNA was extracted and sequenced by Beijing Genomics Institute (BGI, San Jose, CA). The quality of RNA prior to extraction was adequate for each cell line (DV<200%). HiSeq 4000 was used for paired-end sequencing with 40 million reads for RNA. Raw FASTQ sequencing files were run through our in-house computational pipeline.

### Statistical Analysis

For the murine in vivo xenograft study survival analysis, the tumor endpoint volumes for time-to-event (TTE) analysis were set at 0.75 cc and were collected to 1.5 cc. TTE was defined in days by selecting the day in which the tumor volume equaled or surpassed 0.75 cc. Animals that did not reach endpoint volume were assigned a TTE of 21 days for the HB243 analysis and a TTE of 12 days for the G2 analysis. The Kaplan-Meier survival plot represents the percentage of animals surviving at different time points during the study. These percentages were generated from the TTE data using GraphPad Prism 9.0 software (Graphpad Software, San Diego, CA, https://www.graphpad.com/scientific-software/prism/). Survival curve comparisons were analyzed using the Mantel-Cox and Gehan-Breslow-Wilcox tests (95% CI) through Graph Pad Prism 9.0 software. For the quail xenograft assay, significance was determined by an unpaired two-tailed t-test with Welch's correction and a p-value less than 0.05 was considered statistically significant. Error bars represent±standard error of the mean (SEM).

### Acknowledgments

Funding for this work was supported by the Macy Easom Cancer Research Foundation, as well as The Foundation for Addie's Research, Owls for Avery Foundation, Rutledge Foundation, the Super Sam Foundation, the Go4TheGoal Foundation and the Sam Day Foundation.

Histology for this work was done by the OHSU Histopathology Shared Resource.

### Author Contributions

Overall conception and design: CK

Analysis and interpretation of experimental data: CK, SVR, LHP, AM, SR

Development of experimental methodology: CK, SVR, NEB

Provision of experimental reagents: SC, SVR

Acquisition of experimental data: SVR, NEB, SR

Writing, review and/or revision: CK, SVR, SR, SC

Study supervision: CK

### Conflicting Interests

The authors declare no conflict of interests.

### REFERENCES

1. Abraham, J., et al. Evasion mechanisms to Igf1r inhibition in rhabdomyosarcoma. Mol Cancer Ther 10, 697-707 (2011).
2. Aslam, M. I., et al. PDGFRbeta reverses EphB4 signaling in alveolar rhabdomyosarcoma. Proc Natl Acad Sci USA 111, 6383-6388 (2014).
3. Lokman, N. A., Elder, A. S., Ricciardelli, C. & Oehler, M. K. Chick chorioallantoic membrane (CAM) assay as an in vivo model to study the effect of newly identified molecules on ovarian cancer invasion and metastasis. Int J Mol Sci 13, 9959-9970 (2012).
4. Subauste, M. C., et al. Evaluation of metastatic and angiogenic potentials of human colon carcinoma cells in chick embryo model systems. Clin Exp Metastasis 26, 1033-1047 (2009).
5. Handler, A.H., Geiser, C.F. & Farber, S. Studies on mortality in chick embryos resulting from implantation of whole blood and blood fractions from patients and animals with neoplastic disease. Proc Natl Acad Sci USA 48, 1549-1573 (1962).
6. Ainsworth, S. J., Stanley, R. L. & Evans, D. J. Developmental stages of the Japanese quail. J Anat 216, 3-15 (2010).
7. Deryugina, E.I. & Quigley, J.P. Chick embryo chorioallantoic membrane model systems to study and visualize human tumor cell metastasis. Histochem Cell Biol 130, 1119-1130 (2008).
8. Atsushi Kato, D. M., Hiroshi Kagami, Yusuke Atsumi, Shusei Mizushima, Kiyoshi Shimada and Tamao Ono. <Culture System for Bobwhite Quail Embryos from the Blastoderm Stage to Hatching.pdf>. Journal of Poultry Science, 155-158 (2013).
9. Vargas, A., Zeisser-Labouebe, M., Lange, N., Gurny, R. & Delie, F. The chick embryo and its chorioallantoic membrane (CAM) for the in vivo evaluation of drug delivery systems. Adv Drug Deliv Rev 59, 1162-1176 (2007).
10. Kats, D., et al. Volasertib preclinical activity in high-risk hepatoblastoma. Oncotarget 10, 6403-6417 (2019).
11. Lopez-Terrada, D., Cheung, S. W., Finegold, M. J. & Knowles, B. B. Hep G2 is a hepatoblastoma-derived cell line. Hum Pathol 40, 1512-1515 (2009).
12. Stebbing, J., et al. Patient-derived xenografts for individualized care in advanced sarcoma. Cancer 120, 2006-2015 (2014).
13. Fox, E., et al. A phase 1 trial and pharmacokinetic study of cediranib, an orally bioavailable pan-vascular endothelial growth factor receptor inhibitor, in children and adolescents with refractory solid tumors. Journal of clinical oncology: official journal of the American Society of Clinical Oncology 28, 5174-5181 (2010).
14. Broniscer, A., et al. Phase I and pharmacokinetic studies of erlotinib administered concurrently with radiotherapy for children, adolescents, and young adults with high-grade glioma. Clinical cancer research: an official journal of the American Association for Cancer Research 15, 701-707 (2009).
15. Mueller, C. A., Burggren, W. W. & Tazawa, H. Sturkie's Avian Physiology, (Elsevier, New York, 2015).

### Supplementary Information

FIG. 19 (panels A-C) illustrates Cell Number vs luminescence for HepG2Glo tumor modules. Panel A) Lentiviral particle map used for transfecting HepG2. Panel B) Linear relationship for bioluminescent vs HepG2 RFP cell number (R2-0.927). Panel C) The pseudo-colorized images of the multi-well plate holding the tumor modules with number of cells for the bioluminescence in panel B.

FIG. 20 (panels A-B) illustrates the diffusion of IR820 small molecule dye from tumor modules. Panel A) Tumor modules with varying concentrations of IR820 on a log-log scale and n=4 per concentration. Panel B) The diffusion of IR820 into a multi-well plate from a tumor module with a P-10 drug depot beads applied atop of the scaffold.

FIG. 21 (panels A-D) illustrates ex ovo patient-derived xenograft. Panel A is the process for xenografts. Panel B is an Ewing sarcoma mouse PDX explant tumor engraftment. “T” within the panel is in the Ewing sarcoma tumor tissue. “B” within the panel represents nucleated red blood cells in the vasculature from the quail that has grown into the tumor. Panels C-D are autopsy-derived xenograft with vascularization.

TABLE 1

| | | | Chick | | | |
|---|---|---|---|---|---|---|
| | | | Blood | | Quail | Quail |
| | | | Volume | | Embryo | Blood |
| | | Chick | From | | Mass | Volume |
| H&H | Incubation | Embryo | Reference | Incubation | Measured | Calculated |
| Stage | Day Chick | Mass (g) | (mL) | Day Quail | (g) | (mL) |
| 32 | 7 | 0.93 | 0.31 | 7 | 0.52 | 0.13 |
| 35 | 9 | 2.17 | 0.49 | 8 | 0.66 | 0.19 |
| 36 | 10 | 3.17 | 0.62 | 9 | 0.94 | 0.21 |
| 38 | 12 | 6.11 | 1.07 | 10 | 1.46 | 0.28 |
| 40 | 14 | 10.32 | 1.98 | 11 | 1.81 | 0.36 |
| 42 | 16 | 16.10 | 3.24 | 12 | 2.31 | 0.47 |

FIG. 22 illustrates blood compartments of quail embryos and total blood volume for quail eggs. Table 1 shows quail embryo blood volumes calculated proportionally from the ratio of mass to blood volume of a chick for Hamburger and Hamilton developmental stages¹⁵. We assumed that the ratio of blood volume to body mass would be the same during the same growth stages.

TABLE 2
RNA espression levels in TPM for hepatoblastoma cell lines.

| Gene | Normal | HepG2 | HB243 | HB282 |
|---|---|---|---|---|
| PLK1 | 1.45 | 69.26 | 47.88 | 31.45 |
| PLK2 | 36.22 | 6.15 | 42.67 | 107.50 |
| PLK3 | 7.99 | 5.39 | 2.14 | 6.67 |
| ABCB1 | 10.52 | 37.14 | 49.07 | 157.00 |
| ABCC2 | 59.14 | 86.07 | 123.00 | 84.96 |
| ABCC3 | 26.53 | 13.28 | 39.74 | 29.49 |
| ABCC6 | 27.99 | 3.07 | 5.28 | 2.41 |
| ABCG2 | 14.64 | 9.79 | 3.34 | 5.89 |

1. An egg opener for a predetermined number of eggs comprising:
A. a frame, the frame comprising a predetermined number of egg holders, each egg holder equipped for securing a single egg; and
B. a sliding blade holder slidable along the frame, the sliding blade holder comprising a blade; or a predetermined number of blades, and
wherein when the sliding blade holder slides along the frame, the blade cuts each egg in the egg holders,
optionally wherein when the blade cuts each egg, the sliding blade holder collects the egg shell fragments; and
optionally further comprising a standardized multi-well cell culture plate affixed to the egg opener, wherein the alignment and location of the predetermined number of egg holders corresponds to the alignment and location of the wells of the standardized multi-well cell culture plate, and wherein when the blade cuts the eggs the egg contents transfer to the plurality of wells and optionally wherein the standardized multi-well plate is removably-affixed to the egg opener.

2. The egg opener according to claim 1, wherein the predetermined number of egg holders are each sized to hold an egg selected from the group consisting of an avian egg; or a chicken egg, a quail egg and a combination thereof; or a quail egg; or a Japanese quail (Coturnix japonica) egg.

3. A method for preparing an ex ovo tumor xenograft comprising the steps of:
A. preparing an ex ovo CAM; and
B. adding a cell-infused media to the CAM, to form an ex ovo tumor xenograft,
wherein the cell-infused media comprises tumor cells, optionally wherein the tumor cells are suspended in a media; or a gel; or a hydrogel and a combination thereof, to form the cell-infused media, and
optionally further comprising the step of creating a superficial injury to the CAM and placing the cell-infused media in contact with the superficial injury; and
optionally comprising an experimental incubation wherein the CAM is incubated for from 0 hours to about 144 hours after adding the cell-infused media to the CAM.

4. The method for preparing an ex ovo tumor xenograft according to claim 3, further comprising the step of adding the cell-infused media to a scaffold prior to adding the cell-infused media to the CAM.

5. An assay for testing a therapeutic compound comprising the steps of:
A. providing an ex ovo tumor xenograft according to the method according to claim 3; and
B. adding a therapeutic compound to the CAM.

6. The assay for testing a therapeutic compound according to claim 5, further comprising the steps of:
placing a ring around the scaffold;
adding the therapeutic compound into the ring, optionally wherein the therapeutic compound is provided in a delivery media; or wherein the delivery media is selected form the group consisting of a gel, a solid, a liquid and a combination thereof; or a gel, a solid and a combination thereof; or a solid; or a bead; or an osmotic bead;
optionally comprising an experimental incubation step wherein the CAM is incubated for from 0 hours to about 144 hours after adding the cell-infused media to the CAM; and
optionally adding a marker to the CAM and detecting the marker.

7. A method for testing a therapeutic compound via an ex ovo quail egg xenograft assay comprising the steps of:
A. transferring a quail embryo to a well in a standardized multi-well cell culture plate;
B. subjecting the embryo to a CAM formation incubation of from about 60 hours to about 144 hours to develop a CAM, wherein the CAM comprises a CAM surface;
C. culturing at least one cell to form a plurality of cells;
D. suspending the plurality of cells in a media, optionally a gel or a hydrogel, to form a cell-infused media;
E. adding the cell-infused media to a scaffold;
F. creating a superficial injury on the CAM surface;
G. placing a ring on the CAM surface, wherein the ring at least partially encloses the superficial injury;
H. placing the scaffold on the CAM surface in contact with the superficial injury, wherein the scaffold is placed within the ring;
I. combining a delivery media with a therapeutic compound;
J. placing the delivery media in the ring and on top of the scaffold;
K. subjecting the CAM to an experimental incubation; or wherein the experimental incubation is from about 0 hours to about 144 hours;
L. administering a marker to a marker location selected from the well, the embryo, the CAM and a combination thereof; optionally wherein the marker is a luminescent marker, a radioactive marker, a fluorescent marker, and a combination thereof; a luminescent marker, a fluorescent marker; and a combination thereof; or a bioluminescent marker; or a fluorescent marker; and
M. detecting the marker to generate experimental data.

8. The method for testing a therapeutic compound via an ex ovo quail egg xenograft assay according to claim 7, wherein the plurality of cells are a tumor cell; or a liver carcinoma cell; or a hepatoblastoma cell; or a HepG2 human liver hepatoblastoma cancer cells (ATCC HB-8065); or a Ewing sarcoma cell, or a HB234 cell; or a HB282 cell; or a rhabdomyosarcoma cell; or a U48484 (a.k.a., u48484) rhabdomyosarcoma cell; or a SF8628 cell; or a BT474 cell; or a B16F10 cell.

9. The method for testing a therapeutic compound via an ex ovo quail egg xenograft assay according to claim 8, further comprising the step of analyzing the experimental data.

10. A method for treating Ewing sarcoma comprising the step of administering to a patient an effective amount of a VEGFR inhibitor, a poly-kinase inhibitor, a EGFR inhibitor, and a combination thereof; or cediranib, erlotinib, and a combination thereof.

11. A method for treating rhabdomyosarcoma comprising the step of administering to a patient an effective amount of a PI3K/mTOR inhibitor and a combination thereof; or BEZ235.

12. A method for treating hepatoblastoma comprising the step of administering to a patient an effective amount of a PLK inhibitor and a combination thereof; or PLK1, volasertib and a combination thereof.
